SEQUENCE LISTING (104 sequences)

SEQ ID NO: 1 - protein, 609 aa, Homo sapiens [sequence omitted]

SEQ ID NO: 2 - protein, 608 aa, Mus musculus
  REGION 1..608: note = misc_feature - Murine serum albumin (MSA); UniProtKB/ Swiss-Prot: P07724.3; mature form: amino acids 25-608
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 3 - protein, 608 aa, Macaca fascicularis
  REGION 1..608: note = misc_feature - Cynomolgus monkey serum albumin (CSA); mature form: amino acids 25-608
MKWVTFISLLFLFSSAYSRGVFRRDTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKFVAASQAALA

SEQ ID NO: 4 - protein, 608 aa, Rattus norvegicus
  REGION 1..608: note = misc_feature - Rat serum albumin (RSA); UniProtKB/ Swiss-Prot: P02770.2; mature form: amino acids 25-608
MKWVTFLLLLFISGSAFSRGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 5 - protein, 10 aa, synthetic construct
  REGION 1..10: note = Synthetic: 4A01, Hz4A01v1, Hz4A01v3, Hz4A01v4, Hz4A01v5, Hz4A01v6, Hz4A01v8, Hz4A01v9, Hz4A01v3-5, Hz4A01v3-7, Hz4A01v3-8 CDR1
GFAFSTSGMV

SEQ ID NO: 6 - protein, 10 aa, synthetic construct
  REGION 1..10: note = Synthetic: Hz4A01v3-1, Hz4A01v3-7.1k-RITA, Hz4A01v3-7.1k-RITI, Hz4A01v3-7.1k-RITL, Hz4A01v3-7.1k-RITS, Hz4A01v3-7.1k-RITT, Hz4A01v3-7.1k-RITV, Hz4A01v51, Hz4A01v51.9, Hz4A01v51.11, Hz4A01v51.12, Hz4A01v51.13 CDR1
GFTFSTSGMV

SEQ ID NO: 7 - protein, 10 aa, synthetic construct
  REGION 1..10: note = Synthetic: Hz4A01v3-3 CDR1
GFAFSTSGMA

SEQ ID NO: 8 - protein, 10 aa, synthetic construct
  REGION 1..10: note = Synthetic: Hz4A01v3-4 CDR1
GFAFSTSGMS

SEQ ID NO: 9 - protein, 10 aa, synthetic construct
  REGION 1..10: note = Synthetic: 4A01, Hz4A01v1 CDR2
TISDDSRIIR

SEQ ID NO: 10 - protein, 10 aa, synthetic construct
  REGION 1..10: note = Synthetic: Hz4A01v4 CDR2
TISDESRIIR

SEQ ID NO: 11 - protein, 10 aa, synthetic construct
  REGION 1..10: note = Synthetic: Hz4A01v5 CDR2
TISDEGRIIR

SEQ ID NO: 12 - protein, 10 aa, synthetic construct
  REGION 1..10: note = Synthetic: Hz4A01v6 CDR2
TISDSGRIIR

SEQ ID NO: 13 - protein, 10 aa, synthetic construct
  REGION 1..10: note = Synthetic: Hz4A01v8 CDR2
TISDNARIIR

SEQ ID NO: 14 - protein, 10 aa, synthetic construct
  REGION 1..10: note = Synthetic: Hz4A01v9 CDR2
TISDDTRIIR

SEQ ID NO: 15 - protein, 10 aa, synthetic construct
  REGION 1..10: note = Synthetic: Hz4A01v3 Hz4A01v3-1, Hz4A01v3-3, Hz4A01v3-4, Hz4A01v3-5, Hz4A01v3-7, Hz4A01v3-8, CDR2
TISDDARIIR

SEQ ID NO: 16 - protein, 10 aa, synthetic construct
  REGION 1..10: note = Synthetic: Hz4A01v3-7.1k-RITA CDR2
TISDDARITA

SEQ ID NO: 17 - protein, 10 aa, synthetic construct
  REGION 1..10: note = Synthetic: Hz4A01v3-7.1k-RITI CDR2
TISDDARITI

SEQ ID NO: 18 - protein, 10 aa, synthetic construct
  REGION 1..10: note = Synthetic: Hz4A01v3-7.1k-RITL CDR2
TISDDARITL

SEQ ID NO: 19 - protein, 10 aa, synthetic construct
  REGION 1..10: note = Synthetic: Hz4A01v3-7.1k-RITS CDR2
TISDDARITS

SEQ ID NO: 20 - protein, 10 aa, synthetic construct
  REGION 1..10: note = Synthetic: Hz4A01v3-7.1k-RITT CDR2
TISDDARITT

SEQ ID NO: 21 - protein, 10 aa, synthetic construct
  REGION 1..10: note = Synthetic: Hz4A01v3-7.1k-RITV, Hz4A01v51, Hz4A01v51.9, Hz4A01v51.11, Hz4A01v51.12, Hz4A01v51.13 CDR2
TISDDARITV

SEQ ID NO: 22 - protein, 14 aa, synthetic construct
  REGION 1..14: note = Synthetic: 4A01, Hz4A01v1, Hz4A01v3, Hz4A01v4, Hz4A01v5, Hz4A01v6, Hz4A01v8, Hz4A01v9, Hz4A01v3-1, Hz4A01v3-3, Hz4A01v3-4, Hz4A01v3-5, Hz4A01v3-7, Hz4A01v3-8, Hz4A01v3-7.1k-RITA, Hz4A01v3-7.1k-RITI, Hz4A01v3-7.1k-RITL, Hz4A01v3-7.1k-RITS, Hz4A01v3-7.1k-RITT, Hz4A01v3-7.1k-RITV, Hz4A01v51, Hz4A01v51.9, Hz4A01v51.11, Hz4A01v51.12, Hz4A01v51.13 CDR3
YRAGSRIGSYEDYY

SEQ ID NO: 23 - protein, 123 aa, synthetic construct
  REGION 1..123: note = Synthetic: 4A01 VHH
QVQLQESGGGLVQPGGSLRLSCAASGFAFSTSGMVWVRQATGKGLEWVSTISDDSRIIRYADSVNGRFTISRDNAENTLYLLMNSLKPEDTAVYYCATYRAGSRIGSYEDYYRGQGTQVTVSS

SEQ ID NO: 24 - protein, 123 aa, synthetic construct
  REGION 1..123: note = Synthetic: Hz4A01v1
EVQLVESGGGEVQPGGSLRLSCAASGFAFSTSGMVWVRQAPGKGLEWVSTISDDSRIIRYAESVKGRFTISRDNAKNTLYLQMNSLRAEDTAVYYCATYRAGSRIGSYEDYYRGQGTLVTVKP

SEQ ID NO: 25 - protein, 123 aa, synthetic construct
  REGION 1..123: note = Synthetic: Hz4A01v3
EVQLVESGGGEVQPGGSLRLSCAASGFAFSTSGMVWVRQAPGKGLEWVSTISDDARIIRYAESVKGRFTISRDNAKNTLYLQMNSLRAEDTAVYYCATYRAGSRIGSYEDYYRGQGTLVTVKP

SEQ ID NO: 26 - protein, 123 aa, synthetic construct
  REGION 1..123: note = Synthetic: Hz4A01v4
EVQLVESGGGEVQPGGSLRLSCAASGFAFSTSGMVWVRQAPGKGLEWVSTISDESRIIRYAESVKGRFTISRDNAKNTLYLQMNSLRAEDTAVYYCATYRAGSRIGSYEDYYRGQGTLVTVKP

SEQ ID NO: 27 - protein, 123 aa, synthetic construct
  REGION 1..123: note = Synthetic: Hz4A01v5
EVQLVESGGGEVQPGGSLRLSCAASGFAFSTSGMVWVRQAPGKGLEWVSTISDEGRIIRYAESVKGRFTISRDNAKNTLYLQMNSLRAEDTAVYYCATYRAGSRIGSYEDYYRGQGTLVTVKP

SEQ ID NO: 28 - protein, 123 aa, synthetic construct
  REGION 1..123: note = Synthetic: Hz4A01v6
EVQLVESGGGEVQPGGSLRLSCAASGFAFSTSGMVWVRQAPGKGLEWVSTISDSGRIIRYAESVKGRFTISRDNAKNTLYLQMNSLRAEDTAVYYCATYRAGSRIGSYEDYYRGQGTLVTVKP

SEQ ID NO: 29 - protein, 123 aa, synthetic construct
  REGION 1..123: note = Synthetic: Hz4A01v8
EVQLVESGGGEVQPGGSLRLSCAASGFAFSTSGMVWVRQAPGKGLEWVSTISDNARIIRYAESVKGRFTISRDNAKNTLYLQMNSLRAEDTAVYYCATYRAGSRIGSYEDYYRGQGTLVTVKP

SEQ ID NO: 30 - protein, 123 aa, synthetic construct
  REGION 1..123: note = Synthetic: Hz4A01v9
EVQLVESGGGEVQPGGSLRLSCAASGFAFSTSGMVWVRQAPGKGLEWVSTISDDTRIIRYAESVKGRFTISRDNAKNTLYLQMNSLRAEDTAVYYCATYRAGSRIGSYEDYYRGQGTLVTVKP

SEQ ID NO: 31 - protein, 123 aa, synthetic construct
  REGION 1..123: note = Synthetic: Hz4A01v3-1
EVQLVESGGGEVQPGGSLRLSCAASGFTFSTSGMVWVRQAPGKGLEWVSTISDDARIIRYAESVKGRFTISRDNAKNTLYLQMNSLRAEDTAVYYCATYRAGSRIGSYEDYYRGQGTLVTVKP

SEQ ID NO: 32 - protein, 123 aa, synthetic construct
  REGION 1..123: note = Synthetic: Hz4A01v3-3
EVQLVESGGGEVQPGGSLRLSCAASGFAFSTSGMAWVRQAPGKGLEWVSTISDDARIIRYAESVKGRFTISRDNAKNTLYLQMNSLRAEDTAVYYCATYRAGSRIGSYEDYYRGQGTLVTVKP

SEQ ID NO: 33 - protein, 123 aa, synthetic construct
  REGION 1..123: note = Synthetic: Hz4A01v3-4
EVQLVESGGGEVQPGGSLRLSCAASGFAFSTSGMSWVRQAPGKGLEWVSTISDDARIIRYAESVKGRFTISRDNAKNTLYLQMNSLRAEDTAVYYCATYRAGSRIGSYEDYYRGQGTLVTVKP

SEQ ID NO: 34 - protein, 123 aa, synthetic construct
  REGION 1..123: note = Synthetic: Hz4A01v3-5
EVQLVESGGGEVQPGGSLRLSCAASGFAFSTSGMVWYRQAPGKGLEWVSTISDDARIIRYAESVKGRFTISRDNAKNTLYLQMNSLRAEDTAVYYCATYRAGSRIGSYEDYYRGQGTLVTVKP

SEQ ID NO: 35 - protein, 123 aa, synthetic construct
  REGION 1..123: note = Synthetic: Hz4A01v3-7
EVQLVESGGGEVQPGGSLRLSCAASGFAFSTSGMVWVRQAPGKGLEWVSTISDDARIIRYAESVKGRFTISRDNAKNTVYLQMNSLRAEDTAVYYCATYRAGSRIGSYEDYYRGQGTLVTVKP

SEQ ID NO: 36 - protein, 123 aa, synthetic construct
  REGION 1..123: note = Synthetic: Hz4A01v3-8
EVQLVESGGGEVQPGGSLRLSCAASGFAFSTSGMVWVRQAPGKGLEWVSTISDDARIIRYAESVKGRFTISRDNAKNTLYLQMNSLRAEDTAVYYCATYRAGSRIGSYEDYYWGQGTLVTVKP

SEQ ID NO: 37 - protein, 123 aa, synthetic construct
  REGION 1..123: note = Synthetic: Hz4A01v3-7.1k-RITA
EVQLVESGGGEVQPGKSLRLSCAASGFTFSTSGMVWVRQAPGKGLEWVSTISDDARITAYAESVKGRFTISRDNAKNTVYLQMNSLRAEDTAVYYCATYRAGSRIGSYEDYYRGQGTLVTVKP

SEQ ID NO: 38 - protein, 123 aa, synthetic construct
  REGION 1..123: note = Synthetic: Hz4A01v3-7.1k-RITI
EVQLVESGGGEVQPGKSLRLSCAASGFTFSTSGMVWVRQAPGKGLEWVSTISDDARITIYAESVKGRFTISRDNAKNTVYLQMNSLRAEDTAVYYCATYRAGSRIGSYEDYYRGQGTLVTVKP

SEQ ID NO: 39 - protein, 123 aa, synthetic construct
  REGION 1..123: note = Synthetic: Hz4A01v3-7.1k-RITL
EVQLVESGGGEVQPGKSLRLSCAASGFTFSTSGMVWVRQAPGKGLEWVSTISDDARITLYAESVKGRFTISRDNAKNTVYLQMNSLRAEDTAVYYCATYRAGSRIGSYEDYYRGQGTLVTVKP

SEQ ID NO: 40 - protein, 123 aa, synthetic construct
  REGION 1..123: note = Synthetic: Hz4A01v3-7.1k-RITS
EVQLVESGGGEVQPGKSLRLSCAASGFTFSTSGMVWVRQAPGKGLEWVSTISDDARITSYAESVKGRFTISRDNAKNTVYLQMNSLRAEDTAVYYCATYRAGSRIGSYEDYYRGQGTLVTVKP

SEQ ID NO: 41 - protein, 123 aa, synthetic construct
  REGION 1..123: note = Synthetic: Hz4A01v3-7.1k-RITT
EVQLVESGGGEVQPGKSLRLSCAASGFTFSTSGMVWVRQAPGKGLEWVSTISDDARITTYAESVKGRFTISRDNAKNTVYLQMNSLRAEDTAVYYCATYRAGSRIGSYEDYYRGQGTLVTVKP

SEQ ID NO: 42 - protein, 123 aa, synthetic construct
  REGION 1..123: note = Synthetic: Hz4A01v3-7.1k-RITV
EVQLVESGGGEVQPGKSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKP

SEQ ID NO: 43 - protein, 123 aa, synthetic construct
  REGION 1..123: note = Synthetic: Hz4A01v51 VHH
EVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKP

SEQ ID NO: 44-46 - skipped (3 entries)

SEQ ID NO: 47 - protein, 227 aa, synthetic construct
  REGION 1..227: note = Synthetic: human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 224 aa, synthetic construct
  REGION 1..224: note = Synthetic: human IgG1 Fc xELL
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 227 aa, synthetic construct
  REGION 1..227: note = Synthetic: Fc region M252Y and M428V (YV) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGK

SEQ ID NO: 50 - protein, 227 aa, synthetic construct
  REGION 1..227: note = Synthetic: Fc region M252Y, M428V, H435R (YVR) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPGK

SEQ ID NO: 51 - protein, 224 aa, synthetic construct
  REGION 1..224: note = Synthetic: Fc region xELL H435R
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 52 - protein, 224 aa, synthetic construct
  REGION 1..224: note = Synthetic: Fc region xELL M252Y and M428V (YV)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPGK

SEQ ID NO: 53 - protein, 224 aa, synthetic construct
  REGION 1..224: note = Synthetic: Fc region xELL M252Y and M428L (YL)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 54 - protein, 224 aa, synthetic construct
  REGION 1..224: note = Synthetic: Fc region xELL M252Y, M428L, H435R (YLR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPGK

SEQ ID NO: 55 - protein, 224 aa, synthetic construct
  REGION 1..224: note = Synthetic: Fc region xELL M252Y, M428V, H435R (YVR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPGK

SEQ ID NO: 56 - protein, 224 aa, synthetic construct
  REGION 1..224: note = Synthetic: Fc region xELL S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 224 aa, synthetic construct
  REGION 1..224: note = Synthetic: Fc region xELL H435R S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 58 - protein, 224 aa, synthetic construct
  REGION 1..224: note = Synthetic: Fc region xELL M252Y and M428V (YV) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGK

SEQ ID NO: 59 - protein, 224 aa, synthetic construct
  REGION 1..224: note = Synthetic: Fc region xELL M252Y and M428L (YL) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 60 - protein, 224 aa, synthetic construct
  REGION 1..224: note = Synthetic: Fc region xELL M252Y, M428L, H435R (YLR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPGK

SEQ ID NO: 61 - protein, 224 aa, synthetic construct
  REGION 1..224: note = Synthetic: Fc region xELL M252Y, M428V, H435R (YVR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPGK

SEQ ID NO: 62 - protein, 224 aa, synthetic construct
  REGION 1..224: note = Synthetic: Fc region xELL T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 224 aa, synthetic construct
  REGION 1..224: note = Synthetic: Fc region xELL H435R, T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 64 - protein, 224 aa, synthetic construct
  REGION 1..224: note = Synthetic: Fc region xELL M252Y and M428V (YV) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPGK

SEQ ID NO: 65 - protein, 224 aa, synthetic construct
  REGION 1..224: note = Synthetic: Fc region xELL M252Y and M428L (YL) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGK

SEQ ID NO: 66 - protein, 224 aa, synthetic construct
  REGION 1..224: note = Synthetic: Fc region xELL M252Y, M428L, H435R (YLR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPGK

SEQ ID NO: 67 - protein, 224 aa, synthetic construct
  REGION 1..224: note = Synthetic: Fc region xELL M252Y, M428V, H435R (YVR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPGK

SEQ ID NO: 68 - protein, 211 aa, synthetic construct
  REGION 1..211: note = Synthetic: Fc region S364N, Y407N, and K409T (NNT)
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPG

SEQ ID NO: 69 - protein, 250 aa, synthetic construct
  REGION 1..250: note = Synthetic: cx11383 (B10a.v1 VHH x Hz4A01v51 VHH)
EVQLLESGGGEVQPGGSLRLSCTASGSIANVDMMAWHRQAPGKQREPIAVINDGGSTSYADSVKGRFTISRDNAKNTLYLQMSSLRAEDTAVYYCNTATWRGGERYADWGQGTLVTVKPGSGGGSEVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKPGG

SEQ ID NO: 70 - protein, 250 aa, synthetic construct
  REGION 1..250: note = Synthetic: cx11558 (B10a.v2.1 VHH x Hz4A01v51 VHH)
EVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVKPGSGGGSEVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKPGG

SEQ ID NO: 71 - protein, 250 aa, synthetic construct
  REGION 1..250: note = Synthetic: cx12003 (B10a.v2.2 VHH x Hz4A01v51.9 VHH)
QVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVEPGSGGGSQVQLLESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVEPGG

SEQ ID NO: 72 - protein, 250 aa, synthetic construct
  REGION 1..250: note = Synthetic: cx12006 (B10a.v2.3 VHH x Hz4A01v51.11 VHH
QVQLLESGGGKVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVEPGSGGGSQVQLLESGGGKVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVEPGG

SEQ ID NO: 73 - protein, 250 aa, synthetic construct
  REGION 1..250: note = Synthetic: cx12007 (B10a.v2.4 VHH x Hz4A01v51.12 VHH
EVQLLESGGGKVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVEPGSGGGSEVQLLESGGGKVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVEPGG

SEQ ID NO: 74 - protein, 250 aa, synthetic construct
  REGION 1..250: note = Synthetic: cx12008 (B10a.v2.5 VHH x Hz4A01v51.13 VHH)
EVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVRPGSGGGSEVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVRPGG

SEQ ID NO: 75 - protein, 375 aa, synthetic construct
  REGION 1..375: note = Synthetic: cx11385 (B10a.v1 VHH x B10a.v1 VHH x hz4A01v51 VHH)
EVQLLESGGGEVQPGGSLRLSCTASGSIANVDMMAWHRQAPGKQREPIAVINDGGSTSYADSVKGRFTISRDNAKNTLYLQMSSLRAEDTAVYYCNTATWRGGERYADWGQGTLVTVKPGSGGGSEVQLLESGGGEVQPGGSLRLSCTASGSIANVDMMAWHRQAPGKQREPIAVINDGGSTSYADSVKGRFTISRDNAKNTLYLQMSSLRAEDTAVYYCNTATWRGGERYADWGQGTLVTVKPGSGGGSEVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKPGG

SEQ ID NO: 76 - protein, 375 aa, synthetic construct
  REGION 1..375: note = Synthetic: cx11642 (B10a.v2.1 VHH x B10a.v2.1 VHH x hz4A01v51 VHH)
EVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVKPGSGGGSEVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVKPGSGGGSEVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKPGG

SEQ ID NO: 77 - protein, 244 aa, synthetic construct
  REGION 1..244: note = Synthetic: cx12032 (B10a.v2.1 VHH x hz4A01v51 VHH)
EVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVKPEVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKPGG

SEQ ID NO: 78 - protein, 274 aa, synthetic construct
  REGION 1..274: note = Synthetic: FcRn ECD
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 79 - protein, 119 aa, synthetic construct
  REGION 1..119: note = Synthetic: Beta 2 microglobulin
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 80 - protein, 10 aa, synthetic construct
  REGION 1..10: note = Synthetic: FcRn B10a.v1 CDR1
GSIANVDMMA

SEQ ID NO: 81 - protein, 10 aa, synthetic construct
  REGION 1..10: note = Synthetic: FcRn B10a.v2 CDR1
GSIANVDLMA

SEQ ID NO: 82 - skipped

SEQ ID NO: 83 - protein, 9 aa, synthetic construct
  REGION 1..9: note = Synthetic: FcRn B10a.v1 CDR2
VINDGGSTS

SEQ ID NO: 84 - protein, 9 aa, synthetic construct
  REGION 1..9: note = Synthetic: FcRn B10a.v2 CDR2
VINDAGSTY

SEQ ID NO: 85 - protein, 11 aa, synthetic construct
  REGION 1..11: note = Synthetic: FcRn B10a.v1, B10a.v2 CDR3
ATWRGGERYAD

SEQ ID NO: 86 - protein, 119 aa, synthetic construct
  REGION 1..119: note = Synthetic: FcRn B10a.v1 VHH
EVQLLESGGGEVQPGGSLRLSCTASGSIANVDMMAWHRQAPGKQREPIAVINDGGSTSYADSVKGRFTISRDNAKNTLYLQMSSLRAEDTAVYYCNTATWRGGERYADWGQGTLVTVKP

SEQ ID NO: 87 - protein, 119 aa, synthetic construct
  REGION 1..119: note = Synthetic: FcRn B10a.v2.1 VHH
EVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVKP

SEQ ID NO: 88 - protein, 119 aa, synthetic construct
  REGION 1..119: note = Synthetic: FcRn B10a.v2.2 VHH
QVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVEP

SEQ ID NO: 89 - protein, 119 aa, synthetic construct
  REGION 1..119: note = Synthetic: FcRn B10a.v2.3 VHH
QVQLLESGGGKVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVEP

SEQ ID NO: 90 - protein, 119 aa, synthetic construct
  REGION 1..119: note = Synthetic: FcRn B10a.v2.4 VHH
EVQLLESGGGKVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVEP

SEQ ID NO: 91 - protein, 119 aa, synthetic construct
  REGION 1..119: note = Synthetic: FcRn B10a.v2.5 VHH
EVQLLESGGGEVQPGGSLRLSCAASGSIANVDLMAWHRQAPGKQREPIAVINDAGSTYYAESVKGRFTISRDNAKNTLYLQMNSLRAEDTAVYYCNTATWRGGERYADWGQGTLVTVRP

SEQ ID NO: 92 - protein, 365 aa, Homo sapiens
  REGION 1..365: note = misc_feature - UniProtKB P55899 (FCGRN_HUMAN) (human)
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 93 - protein, 365 aa, Mus musculus
  REGION 1..365: note = misc_feature - UniProtKB Q61559 (FCGRN_MOUSE) (mouse)
MGMPLPWALSLLLVLLPQTWGSETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 94 - protein, 365 aa, Macaca fascicularis
  REGION 1..365: note = misc_feature - UniProtKB Q8SPV9 (FCGRN_MACFA) (Cynomolgus monkey)
MRVPRPQPWALGLLLFLLPGSLGAESHLSLLYHLTAVSSPAPGTPAFWVSGWLGPQQYLSYDSLRGQAEPCGAWVWENQVSWYWEKETTDLRIKEKLFLEAFKALGGKGPYTLQGLLGCELSPDNTSVPTAKFALNGEEFMNFDLKQGTWGGDWPEALAISQRWQQQDKAANKELTFLLFSCPHRLREHLERGRGNLEWKEPPSMRLKARPGNPGFSVLTCSAFSFYPPELQLRFLRNGMAAGTGQGDFGPNSDGSFHASSSLTVKSGDEHHYCCIVQHAGLAQPLRVELETPAKSSVLVVGIVIGVLLLTAAAVGGALLWRRMRSGLPAPWISLRGDDTGSLLPTPGEAQDADSKDINVIPATA

SEQ ID NO: 95 - protein, 336 aa, Rattus norvegicus
  REGION 1..336: note = misc_feature - UniProtKB A0A8I6AFJ8 (A0A8I6AFJ8_RAT) (rat)
MGMSQPGVLLSLLLVLLPQTWGAEPRLPLMYHLAAVSDLSWLGAQQYLTYNNLRQEADPCGAWIWENQVSWYWEKETTDLKSKEQLFLEAIRTLENQINGTFTLQGLLGCELAPDNSSLPTAVFALNGEEFMRFNPRTGNWSGEWPETDIVGNLWMKQPEAARKESEFLLTSCPERLLGHLERGRQNLEWKEPPSMRLKARPGNSGSSVLTCAAFSFYPTGPNGDGSFHAWSLLEVKRGDEHHYQCQVEHEGLAQPLTVDLDSPARSSVPVVGIILGLLLVVVAIAGGVLLWNRMRSGLPAPWLSLSGDDSGDLLPGGNLPPEAEPQGVNAFPATS

SEQ ID NO: 96 - protein, 101 aa, Homo sapiens
  REGION 1..101: note = misc_feature - UniProtKB F5H6I0 (F5H6I0_HUMAN) Human B2m
MSRSVALAVLALLSLSGLEAIQRTPKIQVYSRHPADIEVDLLKNGERIEKVEHSDLSFSKDWSFYLLYYTEFTPTEKDEYACRVNHVTLSQPKIVKWDRDM

SEQ ID NO: 97 - protein, 123 aa, synthetic construct
  REGION 1..123: note = Synthetic: Hz4A01v51.9 VHH
QVQLLESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVEP

SEQ ID NO: 98 - protein, 123 aa, synthetic construct
  REGION 1..123: note = Synthetic: Hz4A01v51.11 VHH
QVQLLESGGGKVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVEP

SEQ ID NO: 99 - protein, 123 aa, synthetic construct
  REGION 1..123: note = Synthetic: Hz4A01v51.12 VHH
EVQLLESGGGKVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVEP

SEQ ID NO: 100 - protein, 123 aa, synthetic construct
  REGION 1..123: note = Synthetic: Hz4A01v51.13 VHH
EVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVRP

SEQ ID NO: 101 - protein, 6 aa, synthetic construct
  REGION 1..6: note = Synthetic: Linker
GSGGGS

SEQ ID NO: 102 - protein, 347 aa, synthetic construct
  REGION 1..347: note = Synthetic: cx5009 (hz4A01v51-NNT-hFc)
EVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKPGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGSGGGGSGGGGS

SEQ ID NO: 103 - protein, 351 aa, synthetic construct
  REGION 1..351: note = Synthetic: cx11956 (hz4A01v51-xELL)
EVQLVESGGGEVQPGGSLRLSCAASGFTFSTSGMVWVRQAPGKGLEWVSTISDDARITVYAESVKGRFTISRDNAKNTVYLQMNSLRAEDTAVYYCATYRAGSRIGSYEDYYRGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 96 aa, synthetic construct
  REGION 1..96: note = Synthetic: VH3-23*04
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYC

(19) United States
(12) Patent Application Publication
(10) Pub. No.: US 2025/0353911 A1
(43) Pub. Date: Nov. 20, 2025

(54) FcRn-Binding Polypeptides and Uses Thereof

(71) Applicant: Inhibrx Biosciences, Inc., La Jolla, CA (US)

(72) Inventors: Andrew Hollands, La Jolla, CA (US); Milton Ma, La Jolla, CA (US); John C. Timmer, San Diego, CA (US); Brendan P. Eckelman, Encinitas, CA (US); Florian Sulzmaier, San Diego, CA (US)

(73) Assignee: Inhibrx Biosciences, Inc., La Jolla, CA (US)

(21) Appl. No.: 18/871,526

(22) PCT Filed: Jun. 9, 2023
(86) PCT No.: PCT/US2023/068168

§ 371 Date: Dec. 4, 2024

Related U.S. Application Data
(60) Provisional application No. 63/351,363, filed on Jun. 11, 2022.
(60) Provisional application No. 63/437,776, filed on Jan. 9, 2023.

(51) Int. Cl. C07K 16/28; A61K 9/00; A61K 39/00; A61P 37/06; C07K 16/18

(52) U.S. Cl. CPC C07K 16/283; A61K 9/0019; A61P 37/06; C07K 16/18; A61K 2039/505; A61K 2039/54; C07K 2317/24; C07K 2317/31; C07K 2317/565; C07K 2317/569; C07K 2317/76; C07K 2317/92; C07K 2317/94

(57) ABSTRACT

Provided herein are VHH-containing polypeptides that bind FcRn. Uses of the VHH-containing polypeptides are also provided.

### CROSS-REFERENCE TO RELATED APPLICATIONS

This application claims the benefit of priority of U.S. Provisional Application No. 63/351,363, filed Jun. 11, 2022, and U.S. Provisional Application No. 63/437,776, filed Jan. 9, 2023; each of which is incorporated by reference herein in its entirety for any purpose.

### INCORPORATION BY REFERENCE OF SEQUENCE LISTING

This application incorporates by reference a Sequence Listing submitted with this application in electronic format entitled 01202-0057-00PCT_Sequence_Listing, created Jun. 8, 2023, which is 54,032 bytes in size.

### FIELD

The present invention relates to FcRn-binding polypeptides, including FcRn- and albumin-binding polypeptides, and methods of using the polypeptides, for example, to treat immunological diseases or disorders.

### BACKGROUND

Plasma proteins are eliminated from circulation by two primary mechanisms: renal filtration of molecules below 60 kDa, and micropinocytosis by endothelial cells. Proteins below the renal threshold are rapidly cleared from circulation resulting in a half-life of 1 day or less, while proteins larger than the renal threshold are primarily cleared through micropinocytosis with half-lives around 3-5 days. Albumin and immunoglobulin G (IgG) are proteins with long plasma half-lives of around 15-30 days due to their large size (66 and 150 kDa respectively) and their ability to recycle from endothelial micropinocytosis through pH dependent binding to the neonatal Fc receptor (FcRn).

Long plasma half-life is useful for therapeutic agents to accurately and precisely control plasma drug concentrations, optimize efficacy while limiting toxicity, and reduce the frequency and amount of drug needed.

Therefore, there exists a need for FcRn-binding polypeptides that have improved plasma half-life.

### SUMMARY

Embodiment 1. A polypeptide comprising at least one VHH domain that binds FcRn, wherein at least one VHH domain that binds FcRn comprises a CDR1 sequence selected from SEQ ID NOs: 80-81, a CDR2 sequence selected from SEQ ID NOs: 83-84, and a CDR3 sequence of SEQ ID NO: 85.

Embodiment 2. The polypeptide of embodiment 1, wherein each VHH domain that binds FcRn comprises, independently, a CDR1 sequence selected from SEQ ID NOs: 80-81, a CDR2 sequence selected from SEQ ID NOs: 83-84, and a CDR3 sequence of SEQ ID NO: 85.

Embodiment 3. The polypeptide of embodiment 1 or embodiment 2, wherein at least one VHH domain that binds FcRn comprises CDR1, CDR2, and CDR3 sequences selected from: SEQ ID NOs: 80, 83, and 85 SEQ ID NOs: 81, 83, and 85; and SEQ ID NOs: 81, 84, and 85.

Embodiment 4. The polypeptide of embodiment 3, wherein each VHH domain that binds FcRn comprises, independently, CDR1, CDR2, and CDR3 sequences selected from: SEQ ID NOs: 80, 83, and 85 SEQ ID NOs: 81, 83, and 85; and SEQ ID NOs: 81, 84, and 85.

Embodiment 5. The polypeptide of any one of embodiments 1-4, wherein at least one VHH domain that binds FcRn is humanized.

Embodiment 6. The polypeptide of embodiment 5, wherein each VHH domain that binds FcRn is humanized.

Embodiment 7. The polypeptide of any one of embodiments 1-6, wherein at least one VHH domain that binds FcRn comprises a sequence at least 85%, at least 90%, at least 95%, at least 96%, at least 97%, at least 98%, at least 99% identical to a sequence selected from SEQ ID NOs: 86-93.

Embodiment 8. The polypeptide of embodiment 7, wherein each VHH domain that binds FcRn comprises a sequence at least 85%, at least 90%, at least 95%, at least 96%, at least 97%, at least 98%, at least 99% identical to a sequence selected from SEQ ID NOs: 86-93.

Embodiment 9. The polypeptide of any one of embodiments 1-7, wherein at least one VHH domain that binds FcRn comprises a sequence selected from SEQ ID NOs: 86-93.

Embodiment 10. The polypeptide of any one of embodiments 1-9, wherein each VHH domain that binds FcRn comprises a sequence selected from SEQ ID NOs: 86-93.

Embodiment 11. The polypeptide of any one of embodiments 1-10, wherein at least one VHH domain that binds FcRn binds human FcRn with an affinity of less than 5 nM, less than 2 nM, less than 1 nM, or less than 0.5 nM.

Embodiment 12. The polypeptide of any one of embodiments 1-11, wherein each VHH domain that binds FcRn binds human FcRn with an affinity of less than 5 nM, less than 2 nM, less than 1 nM, or less than 0.5 nM.

Embodiment 13. The polypeptide of any one of embodiments 1-12, wherein at least one VHH domain that binds FcRn binds human FcRn at pH 6 and at pH 7.4.

Embodiment 14. The polypeptide of any one of embodiments 1-13, wherein each VHH domain that binds FcRn binds human FcRn at pH 6 and at pH 7.4.

Embodiment 15. The polypeptide of any one of embodiments 1-14, wherein at least one VHH domain that binds FcRn blocks binding of human IgG to human FcRn.

Embodiment 16. The polypeptide of any one of embodiments 1-15, wherein each VHH domain that binds FcRn blocks binding of human IgG to human FcRn.

Embodiment 17. The polypeptide of any one of embodiments 1-16, wherein the polypeptide comprises at least one VHH domain that binds albumin.

Embodiment 18. The polypeptide of embodiment 17, wherein at least one VHH domain that binds albumin comprises a CDR1 sequence selected from SEQ ID NOs: 5-8, a CDR2 sequence selected from SEQ ID NOs: 9-21, and a CDR3 sequence of SEQ ID NO: 22.

Embodiment 19. That polypeptide of embodiment 17, wherein each VHH domain that binds albumin comprises, independently, a CDR1 sequence selected from SEQ ID NOs: 5-8, a CDR2 sequence selected from SEQ ID NOs: 9-21, and a CDR3 sequence of SEQ ID NO: 22.

Embodiment 20. The polypeptide of embodiment 17 or embodiment 18, wherein at least one VHH domain that binds albumin comprises CDR1, CDR2, and CDR3 sequences selected from: SEQ ID NOs: 5, 9, and 22; SEQ ID NOs: 5, 10, and 22; SEQ ID NOs: 5, 11, and 22; SEQ ID NOs: 5, 12, and 22; SEQ ID NOs: 5, 13, and 22; SEQ ID NOs: 5, 14, and 22; SEQ ID NOs: 5, 15, and 22; SEQ ID NOs: 6, 15, and 22; SEQ ID NOs: 7, 15, and 22; SEQ ID NOs: 8, 15, and 22; SEQ ID NOs: 6, 16, and 22; SEQ ID NOs: 6, 17, and 22; SEQ ID NOs: 6, 18, and 22; SEQ ID NOs: 6, 19, and 22; SEQ ID NOs: 6, 20, and 22; and SEQ ID NOs: 6, 21, and 22.

Embodiment 21. The polypeptide of embodiment 17 or embodiment 18, wherein each VHH domain that binds albumin comprises, independently, CDR1, CDR2, and CDR3 sequences selected from: SEQ ID NOs: 5, 9, and 22; SEQ ID NOs: 5, 10, and 22; SEQ ID NOs: 5, 11, and 22; SEQ ID NOs: 5, 12, and 22; SEQ ID NOs: 5, 13, and 22; SEQ ID NOs: 5, 14, and 22; SEQ ID NOs: 5, 15, and 22; SEQ ID NOs: 6, 15, and 22; SEQ ID NOs: 7, 15, and 22; SEQ ID NOs: 8, 15, and 22; SEQ ID NOs: 6, 16, and 22; SEQ ID NOs: 6, 17, and 22; SEQ ID NOs: 6, 18, and 22; SEQ ID NOs: 6, 19, and 22; SEQ ID NOs: 6, 20, and 22; and SEQ ID NOs: 6, 21, and 22.

Embodiment 22. The polypeptide of any one of embodiments 17-21, wherein at least one VHH domain that binds albumin is humanized.

Embodiment 23. The polypeptide of embodiment 22, wherein each VHH domain that binds albumin is humanized.

Embodiment 24. The polypeptide of any one of embodiments 17-23, wherein at least one VHH domain that binds albumin comprises a sequence at least 85%, at least 90%, at least 95%, at least 96%, at least 97%, at least 98%, at least 99% identical to a sequence selected from SEQ ID NOs: 23-43 and 97-100.

Embodiment 25. The polypeptide of any one of embodiments 17-23, wherein each VHH domain that binds albumin comprises a sequence at least 85%, at least 90%, at least 95%, at least 96%, at least 97%, at least 98%, at least 99% identical to a sequence selected from SEQ ID NOs: 23-43 and 97-100.

Embodiment 26. The polypeptide of any one of embodiments 17-23, wherein at least one VHH domain that binds albumin comprises a sequence selected from SEQ ID NOs: 23-43 and 97-100.

Embodiment 27. The polypeptide of any one of embodiments 17-23, wherein each VHH domain that binds albumin comprises a sequence selected from SEQ ID NOs: 23-43 and 97-100.

Embodiment 28. The polypeptide of any one of embodiments 17-26, wherein the polypeptide comprises a sequence at least 85%, at least 90%, at least 95%, at least 96%, at least 97%, at least 98%, or at least 99% identical to a sequence selected from SEQ ID NOs: 69-77.

Embodiment 29. The polypeptide of any one of embodiments 17-26, wherein the polypeptide comprises a sequence selected from SEQ ID NOs: 69-77.

Embodiment 30. The polypeptide of any one of embodiments 17-29, wherein at least one VHH domain that binds albumin binds human albumin and at least one albumin selected from cynomolgus monkey, mouse, and rat albumin.

Embodiment 31. The polypeptide of any one of embodiments 17-29, wherein each VHH domain that binds albumin binds human albumin and at least one albumin selected from cynomolgus monkey, mouse, and rat albumin.

Embodiment 32. The polypeptide of any one of embodiments 17-29, wherein at least one VHH domain that binds albumin binds human, cynomolgus monkey, mouse, and rat albumin.

Embodiment 33. The polypeptide of any one of embodiments 17-29, wherein each VHH domain that binds albumin binds human, cynomolgus monkey, mouse, and rat albumin.

Embodiment 34. The polypeptide of any one of embodiments 17-33, wherein at least one VHH domain that binds albumin binds human albumin with an affinity of less than 5 nM, less than 2 nM, less than 1 nM, or less than 0.5 nM.

Embodiment 35. The polypeptide of any one of embodiments 17-34, wherein at least one VHH domain that binds albumin binds each of human, cynomolgus monkey, mouse, and rat albumin with an affinity of less than 5 nM, less than 2 nM, less than 1 nM, or less than 0.5 nM.

Embodiment 36. The polypeptide of any one of embodiments 17-35, wherein each VHH domain that binds albumin binds human albumin with an affinity of less than 5 nM, less than 2 nM, less than 1 nM, or less than 0.5 nM.

Embodiment 37. The polypeptide of any one of embodiments 17-36, wherein each VHH domain that binds albumin binds each of human, cynomolgus monkey, mouse, and rat albumin with an affinity of less than 5 nM, less than 2 nM, less than 1 nM, or less than 0.5 nM.

Embodiment 38. The polypeptide of any one of embodiments 17-37, wherein the each VHH domain that binds albumin does not bind albumin domain 3.

Embodiment 39. The polypeptide of any one of embodiments 17-38, wherein the each VHH domain that binds albumin does not interfere with binding of albumin to FcRn.

Embodiment 40. The polypeptide of any one of the preceding embodiments, wherein the polypeptide comprises at least two, at least three, or at least four VHH domains that bind FcRn and at least one VHH domain that binds albumin.

Embodiment 41. The polypeptide of embodiment 40, wherein the polypeptide comprises two, three, or four VHH domains that bind FcRn and one VHH domain that binds albumin.

Embodiment 42. The polypeptide of any one of embodiments 1-41, wherein the polypeptide comprises at least one binding domain that binds a protein other than albumin or FcRn.

Embodiment 43. The polypeptide of embodiment 42, wherein at least one binding domain that binds a protein other than albumin or FcRn is a VHH.

Embodiment 44. The polypeptide of embodiment 43, wherein each binding domain that binds a protein other than albumin or FcRn is a VHH.

Embodiment 45. The polypeptide of embodiment 42 or embodiment 43, wherein at least one binding domain that binds a protein other than albumin or FcRn comprises a heavy chain variable region and a light chain variable region.

Embodiment 46. The polypeptide of embodiment 45, wherein each binding domain that binds a protein other than albumin or FcRn comprises a heavy chain variable region and a light chain variable region.

Embodiment 47. The polypeptide of any one of embodiments 42-46, wherein at least one binding domain that binds a protein other than albumin or FcRn is a binding domain of a therapeutic antibody.

Embodiment 48. The polypeptide of embodiment 47, wherein each binding domain that binds a protein other than albumin is a binding domain of a therapeutic antibody.

Embodiment 49. The polypeptide of embodiment 47 or embodiment 48, wherein the therapeutic antibody is useful for treating a disease or disorder selected from an autoimmune disease or disorder, an inflammatory disease or disorder, an infection, and cancer.

Embodiment 50. The polypeptide of any one of embodiments 1-41, wherein the polypeptide comprises an amino acid sequence of a therapeutic protein.

Embodiment 51. The polypeptide of embodiment 50, wherein the therapeutic protein is useful for treating a disease or disorder selected from an autoimmune disease or disorder, an inflammatory disease or disorder, an infection, and cancer.

Embodiment 52. The polypeptide of any one of the preceding embodiments, wherein the half-life of the polypeptide is greater than the half-life of the same polypeptide lacking a VHH domain that binds FcRn or albumin.

Embodiment 53. A pharmaceutical composition comprising the polypeptide of any one of embodiments 1-52 and a pharmaceutically acceptable carrier.

Embodiment 54. An isolated nucleic acid that encodes the the polypeptide of any one of embodiments 1-52.

Embodiment 55. A vector comprising the nucleic acid of embodiment 54.

Embodiment 56. A host cell comprising the nucleic acid of embodiment 54 or the vector of embodiment 55.

Embodiment 57. A host cell that expresses the polypeptide of any one of embodiments 1-52.

Embodiment 58. A method of producing the polypeptide of any one of embodiments 1-52, comprising incubating the host cell of embodiment 56 or embodiment 57 under conditions suitable for expression of the polypeptide.

Embodiment 59. The method of embodiment 58, further comprising isolating the antibody or polypeptide.

Embodiment 60. A method comprising administering to a subject the polypeptide of any one of embodiments 1-52 or the pharmaceutical composition of embodiment 53.

Embodiment 61. A method of treating a disease or disorder comprising administering to a subject with the disease or disorder a pharmaceutically effective amount of the polypeptide of any one of embodiments 1-52, or the pharmaceutical composition of embodiment 53.

Embodiment 62. The method of embodiment 61, wherein the disease or disorder is selected from an autoimmune disease or disorder, an inflammatory disease or disorder, an infection, and cancer.

Embodiment 63. The method of embodiment 61 or embodiment 62, wherein the disease or disorder is an autoantibody-mediated disease or disorder.

Embodiment 64. The method of any one of embodiments 61-63, wherein the disease or disorder is pemphigus vulgaris, lupus nephritis, myasthenia gravis, Guillain-Barré syndrome, antibody-mediated rejection, antiphospholipid antibody syndrome, chronic inflammatory demyelinating polyneuropathy, immune complex-mediated vasculitis, glomerulitis, a channelopathy, neuromyelitis optica, autoimmune encephalitis, autoimmune Grave's disease, idiopathic thrombocytopenia purpura, autoimmune haemolytic anaemia, immune neutropenia, dilated cardiomyopathy, or serum sickness.

Embodiment 65. The method of any one of embodiments 60-64, wherein the polypeptide is administered subcutaneously.

### BRIEF DESCRIPTION OF THE FIGURES

FIG. 1A-1I. FIG. 1A shows shows an alignment of certain 4A01 humanized variants (SEQ ID NOs: 24-43). FIG. 1B-1C show human albumin (HSA) binding ELISA of certain 4A01 humanized variants. FIG. 1D-1E show cynomolgus monkey albumin (CSA) binding ELISA of certain 4A01 humanized variants. FIG. 1F-1G show murine albumin (MSA) binding ELISA of certain 4A01 humanized variants. FIG. 1H-1I show rat albumin (RSA) binding ELISA of certain 4A01 humanized variants.

FIG. 2A-2D show human albumin (HSA; FIG. 2A), cynomolgus monkey albumin (CSA; FIG. 2B), murine albumin (MSA; FIG. 2C), and rat albumin (RSA; FIG. 2D) binding ELISA of humanized hz4A01v51.

FIG. 3A-3B. FIG. 3A is a schematic showing the experimental design for biolayer interferometry assessing binding to albumin domain 3. FIG. 3B shows a biolayer interferometry trace showing 4A01-NNT-hFc (SEQ ID NOs: 23 and 68) does not bind to albumin domain 3, while 1C04 does.

FIG. 4A-4B. FIG. 4A is a schematic showing the experimental design for biolayer interferometry assessing B2M-FcRn binding to albumin. FIG. 4B is a biolayer interferometry trace showing 4A01-NNT-hFc (SEQ ID NOs: 23 and 68) and humanized hz4A01v51-NNT-hFc (SEQ ID NO: 102) do not compete with albumin for B2m-FcRn binding, while 1C04 does.

FIG. 5 shows certain exemplary formats tested in binding and functional assays. Formats (i) and (iii) are bivalent, bispecific polypeptides targeting FcRn (black) and albumin (grey). Format (ii) is a trivalent, bi-specific polypeptide consisting of two units targeting FcRn and one unit targeting albumin. Binding units are linked via glycine-serine linkers in format (i) and (ii) and are directly connected in format (iii).

FIG. 6A-6G show binding of multivalent, multispecific polypeptides targeting FcRn and albumin to FcRn transfected CHO cells and cell subpopulations in human peripheral blood (PBMC). FIGS. 6A and 6B show binding to transiently transfected CHO cells at pH 7.4, FIG. 6C shows binding to FcRn by ELISA at pH 7.4, FIGS. 6D and 6E show binding to transiently transfected CHO cells at pH 6. FIG. 6F and FIG. 6G show binding to human PBMC at pH 7.4 and pH 6, respectively. For FIGS. 6A, 6D, 6F and 6G, polypeptides are formatted as described in FIG. 5(i), for FIGS. 6B and 6E, polypeptides are formatted as described in FIG. 5(i) except for cx12032, which is formatted as described in FIG. 5(iii). For FIG. 6C cx11383 is formatted as described in FIG. 5(i) and cx11385 is formatted as described in FIG. 5(ii).

FIG. 7A-7D show binding of multivalent, multispecific polypeptides targeting FcRn and albumin to FcRn to human albumin by ELISA. FIGS. 7A and 7B show binding at pH 7.4, while FIGS. 7C and 7D show binding at pH 5.5. Constructs in FIG. 7 are formatted as described in FIG. 5(i), except for cx12032, which is formatted as described in FIG. 5(iii).

FIG. 8 shows the dose-dependent ability of multivalent, multispecific polypeptides targeting FcRn and albumin to prevent IgG binding to FcRn transfected 293 cells at pH 6. The polypeptides are formatted as described in FIG. 5(i) (cx11383) and FIG. 5(ii) (cx11385).

FIG. 9A-9D show the pharmacokinetic (PK) profile and ability to deplete IgG in vivo of multivalent, multispecific polypeptides targeting FcRn and albumin compared to an internally generated sequence analog of Nipocalimab. FIGS. 9A, 9B and 9C show the serum PK profile in BALB/c mice (FIGS. 9A and 9B) or human FcRn/albumin transgenic C57BL/6 mice (FIG. 9C).

FIG. 9D shows the serum levels of administered human IgG in the transgenic mouse strain after dosing of FcRn blocking test articles. The polypeptides are formatted as described in FIG. 5(i) (cx11558 and cx11383) and FIG. 5(ii) (cx11642 and cx11385).

FIG. 10A-10C show the results of an in vivo study in cynomolgus monkeys after dosing with FcRn blocking test articles cx11558 or cx11642 at 100 mg/kg and 20 mg/kg or the recombination engineered IgG Fc fragment efgartigimod at 20 mg/kg. FIG. 10A shows the serum levels of cynomolgus IgG, plotted as % change from baseline (indicated by the dotted line). FIG. 10B shows the receptor occupancy. FIG. 10C shows the serum levels of cx11558, cx11642, and efgartigimod (the lower limit of quantitation for each test article is indicated by a dotted line).

FIG. 11A-11C show the results of an in vivo study in cynomolgus monkeys after dosing with the multispecific polypeptide cx12007 targeting FcRn and albumin at 100 mg/kg or 20 mg/kg I.V. or 100 mg/kg S.C. FIG. 11A shows the serum levels of cynomolgus IgG, plotted as % change from baseline (indicated by the dotted line). FIG. 11B shows the receptor occupancy.

FIG. 11C shows the serum levels of cx12007 (the lower limit of quantitation is indicated by a dotted line).

FIG. 12A-12B show the results of an in vivo study in cynomolgus monkeys after dosing with the multispecific polypeptide cx12007 targeting FcRn and albumin at 20 mg/kg, 40 mg/kg, or 70 mg/kg S.C. FIG. 12A shows the serum levels of cynomolgus IgG, plotted as % change from baseline (indicated by the dotted line). FIG. 12B shows the serum levels of cx12007 (the lower limit of quantitation is indicated by a dotted line).

### DETAILED DESCRIPTION

Embodiments provided herein relate to FcRn-binding polypeptides, including FcRn- and albumin-binding polypeptides, and uses thereof.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3^{rd} edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgGI EU antibody.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question. In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“Albumin” as used herein refers to any native, mature albumin that results from processing of an albumin precursor in a cell. The term includes albumin from any vertebrate source, including mammals such as primates (e.g., humans and cynomolgus or rhesus monkeys) and rodents (e.g., mice and rats), unless otherwise indicated. The term also includes naturally-occurring variants of albumin, such as splice variants or allelic variants. A nonlimiting exemplary mature human albumin amino acid sequence is shown, e.g., in UniProt Accession No. P02768.2. See SEQ ID NO. 1. Nonlimiting exemplary murine, cynomolgus monkey, and rat albumin amino acid sequences are shown in SEQ ID NOs: 2-4.

“FcRn” as used herein refers to any native, neonatal Fc receptor (FcRn) that results from processing of a FcRn precursor in a cell. The term includes FcRn from any vertebrate source, including mammals such as primates (e.g., humans and cynomolgus or rhesus monkeys) and rodents (e.g., mice and rats), unless otherwise indicated. The term also includes naturally-occurring variants of FcRn, such as splice variants or allelic variants. A nonlimiting exemplary human FcRn amino acid sequence is shown, e.g., in UniProt Accession No. P55899. See SEQ ID NO. 92. Nonlimiting exemplary murine, cynomolgus monkey, and rat FcRn amino acid sequences are shown in SEQ ID NOs: 93-95.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to an FcRn epitope is a sdAb or VHH-containing polypeptide that binds this epitope with greater affinity, avidity, more readily, and/or with greater duration than it binds to other FcRn epitopes or non-FcRn epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, the term “inhibit” with regard to the activity of a target protein refers to a decrease in the activity of the protein. In some embodiments, “inhibit” refers to a decrease in activity compared to the protein in the absence of the modulator.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “antibody” is used in the broadest sense and encompass various polypeptides that comprise antibody-like antigen-binding domains, including but not limited to conventional antibodies (typically comprising at least one heavy chain and at least one light chain), single-domain antibodies (sdAbs, comprising at least one VHH domain and, optionally, an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CHI typically pairs with a light chain constant domain, CL, while the hinge mediates dimerization) and Fc regions (comprising hinge, CH2, and CH3, where the hinge mediates dimerization).

The term antibody also includes, but is not limited to, chimeric antibodies, humanized antibodies, and antibodies of various species such as camelid (including llama), shark, mouse, human, cynomolgus monkey, etc.

The term “antigen-binding domain” as used herein refers to a portion of an antibody sufficient to bind antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FRI and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain of an sdAb or VHH-containing polypeptide comprises three CDRs of a VHH domain. Thus, in some embodiments, an antigen binding domain of an sdAb or VHH-containing polypeptide comprises a VHH domain comprising CDR1-FR2-CDR2-FR3-CDR3, and any portions of FRI and/or FR4 required to maintain binding to antigen.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and optionally an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a a polypeptide having the structure VHH₁-VHH₂, VHH₁-VHH₂-Fc, VHH₁-VHH₂-VHH₃, or VHH₁-VHH₂-VHH₃-Fc, wherein VHH₁, VHH₂, and VHH₃ may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH₁-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, when a VHH-containing polypeptide comprises an Fc, it forms a dimer. Thus, the structure VHH₁-VHH₂-Fc, if it forms a dimer, is considered to be tetravalent (i.e., the dimer has four VHH domains). Similarly, the structure VHH₁-VHH₂-VHH₃-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains, CH1, hinge, CH2, and CH3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is an IgG1, IgG2, IgG3, or IgG4.

An “acceptor human framework” as used herein is a framework comprising the amino acid sequence of a heavy chain variable domain (V_{H}) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D-apparent}.

In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation, and inducing or increasing expression of cytokines.

An “agonist” or “activating” antibody is one that increases and/or activates a biological activity of the target antigen. In some embodiments, the agonist antibody binds to an antigen and increases its biologically activity by at least about 20%, 40%, 60%, 80%, 85% or more.

An “antagonist”, a “blocking” or “neutralizing” antibody is one that inhibits, decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” sdAb or VHH-containing polypeptide refers to a sdAb or VHH-containing polypeptide with one or more alterations in one or more CDRs compared to a parent sdAb or VHH-containing polypeptide that does not possess such alterations, such alterations resulting in an improvement in the affinity of the sdAb or VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18 (12): 592-598 (1997); Ghetie et al., Nature Biotechnology, 15 (7): 637-640 (1997); Hinton et al., J. Biol. Chem. 279 (8): 6213-6216 (2004); WO 2004/92219 (Hinton et al.).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Nonlimiting exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “autoimmune disorder” refers to a disease or disorder typically associated with the nonanaphylactic hypersensitivity reactions (Type II, Type III and/or Type IV hypersensitivity reactions) that generally results from a subject's own humoral and/or cell-mediated immune response to one or more immunogenic substances of endogenous exogenous origin.

The term “inflammatory disorder” refers to disorders associated with inflammation, including, but not limited to, chronic or acute inflammatory diseases, and expressly includes inflammatory autoimmune diseases and inflammatory allergic conditions.

The term “infection” and “infectious disease or disorder” refer to a disease or disorder caused by an exogenous infectious agent, such as, but not limited to, bacteria, viruses, fungi, protozoa, and parasites.

The terms “cancer” and “tumor” encompass solid and hematological/lymphatic cancers and also encompass malignant, pre-malignant, and benign growth, such as dysplasia. Exemplary cancers include, but are not limited to: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effect of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹ In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary FcRn-Binding Polypeptides

Single-domain antibodies (sdAbs) including VHH domains that bind FcRn are provided herein. In some embodiments, a VHH domain that binds FcRn binds with an affinity (K_{D}) between 0.01 nM and 5 nM, or between 0.01 nM at 2 nM, or between 0.01 nM and 1 nM, between 0.01 nM and 0.5 nM, 0.05 nM and 5 nM, or between 0.05 nM at 2 nM, or between 0.05 nM and 1 nM, or between 0.05 nM and 0.5 nM.

In various embodiments, a polypeptide comprising at least one VHH domain that binds FcRn is provided. In some embodiments, a polypeptide comprising one, two, three, four, five, six, seven, or eight VHH domains that bind FcRn is provided. In some embodiments, a polypeptide provided herein comprises one, two, three, or four VHH domains that bind FcRn. Such polypeptides may comprise one or more additional VHH domains that bind one or more target proteins other than FcRn.

In various embodiments, a polypeptide that comprises one or more VHH domains that bind FcRn also comprises a therapeutic antigen-binding domain and/or a therapeutic polypeptide. Such therapeutic antigen-binding domains include, but are not limited to, traditional antibody antigen-binding domains, which comprise a heavy chain variable region and a light chain variable region, and single-domain antibody antigen-binding domains, such as VHH domains. Nonlimiting exemplary therapeutic polypeptides include, for example, receptor extracellular domains, enzymes, and ligands. In various embodiments, the polypeptide comprising at least one VHH domain that binds FcRn has a longer half-life in vivo when fused to at least one VHH domain that binds to albumin than the same polypeptide that is not fused to at least one VHH domain that binds albumin. In some embodiments, the half-life is at least 1.5×, at least 2×, at least 3×, at least 4×, or at least 5× longer than the half-life of the polypeptide without the VHH domain that binds albumin.

In some embodiments, a polypeptide that comprises at least one VHH domain that binds FcRn comprises an Fc region. In some embodiments, a polypeptide provided herein comprises one, two, three, or four VHH domains that bind FcRn and an Fc region. In some embodiments, an Fc region mediates dimerization of the polypeptide at physiological conditions.

In various embodiments, a VHH domain that binds FcRn comprises a CDR1 sequence selected from SEQ ID NOs: 80-81, a CDR2 sequence selected from SEQ ID NOs: 83-84, and a CDR3 sequence of SEQ ID NO: 85. In various embodiments, a VHH domain that binds FcRn comprises CDR1, CDR2, and CDR3 sequences selected from: SEQ ID NOs: 80, 83, and 85; SEQ ID NOs: 81, 83, and 85; and SEQ ID NOs: 81, 84, and 85.

In some embodiments, a VHH domain that binds FcRn comprises an amino acid sequence that is at least 85%, at least 90%, at least 95%, at least 96%, at least 97%, at least 98%, at least 99% identical to a sequence selected from SEQ ID NOs: 86-93. In some embodiments, a VHH domain that binds FcRn comprises an amino acid sequence selected from SEQ ID NOs: 86-93.

In some embodiments, a VHH domain that binds FcRn is provided, which competes for binding to FcRn with a VHH domain comprising an amino acid sequence selected from SEQ ID NOs: 86-93.

### Exemplary Albumin-Binding Polypeptides

Polypeptides including VHH domains that bind albumin are provided herein. In some embodiments, a VHH domain that binds albumin does not interfere with albumin binding to FcRn. In some embodiments, a VHH domain that binds albumin does not bind domain 3 of albumin. In some embodiments, a VHH domain that binds albumin binds with an affinity (K_{D}) between 0.01 nM and 5 nM, or between 0.01 nM at 2 nM, or between 0.01 nM and 1 nM, between 0.01 nM and 0.5 nM, 0.05 nM and 5 nM, or between 0.05 nM at 2 nM, or between 0.05 nM and 1 nM, or between 0.05 nM and 0.5 nM.

In various embodiments, a polypeptide comprising at least one VHH domain that binds albumin is provided. In some embodiments, a polypeptide comprising one, two, three, four, five, six, seven, or eight VHH domains that bind albumin is provided. In some embodiments, a polypeptide provided herein comprises one, two, three, or four VHH domains that bind albumin. Such polypeptides may comprise one or more additional VHH domains that bind one or more target proteins other than albumin.

In various embodiments, a polypeptide that comprises one or more VHH domains that bind albumin also comprises a therapeutic antigen-binding domain and/or a therapeutic polypeptide. Such therapeutic antigen-binding domains include, but are not limited to, traditional antibody antigen-binding domains, which comprise a heavy chain variable region and a light chain variable region, and single-domain antibody antigen-binding domains, such as VHH domains. Nonlimiting exemplary therapeutic polypeptides include, for example, receptor extracellular domains, enzymes, and ligands. In various embodiments, the polypeptide comprising at least one VHH domain that binds albumin has a longer half-life in vivo than the same polypeptide without the at least one VHH domain that binds albumin. In some embodiments, the half-life is at least 1.5×, at least 2×, at least 3×, at least 4×, or at least 5× longer than the half-life of the polypeptide without the VHH domain that binds albumin.

In some embodiments, a polypeptide that comprises at least one VHH domain that binds albumin comprises an Fc region. In some embodiments, a polypeptide provided herein comprises one, two, three, or four VHH domains that bind albumin and an Fc region. In some embodiments, an Fc region mediates dimerization of the polypeptide at physiological conditions.

In various embodiments, a VHH domain that binds albumin comprises a CDR1 sequence selected from SEQ ID NOs: 5-8, a CDR2 sequence selected from SEQ ID NOs: 9-21, and a CDR3 sequence of SEQ ID NO: 22. In various embodiments, a VHH domain that binds albumin comprises CDR1, CDR2, and CDR3 sequences selected from: SEQ ID NOs: 5, 9, and 22; SEQ ID NOs: 5, 10, and 22; SEQ ID NOs: 5, 11, and 22; SEQ ID NOs: 5, 12, and 22; SEQ ID NOs: 5, 13, and 22; SEQ ID NOs: 5, 14, and 22; SEQ ID NOs: 5, 15, and 22; SEQ ID NOs: 6, 15, and 22; SEQ ID NOs: 7, 15, and 22; SEQ ID NOs: 8, 15, and 22; SEQ ID NOs: 6, 16, and 22; SEQ ID NOs: 6, 17, and 22; SEQ ID NOs: 6, 18, and 22; SEQ ID NOs: 6, 19, and 22; SEQ ID NOs: 6, 20, and 22; and SEQ ID NOs: 6, 21, and 22. In some embodiments, a VHH domain that binds albumin comprises CDR1, CDR2, and CDR3 sequences of SEQ ID NOs: 6, 21, and 22.

In some embodiments, a VHH domain that binds albumin comprises an amino acid sequence that is least 85%, at least 90%, at least 95%, at least 96%, at least 97%, at least 98%, at least 99% identical to a sequence selected from SEQ ID NOs: 23-43 and 97-100. In some embodiments, a VHH domain that binds albumin comprises an amino acid sequence selected from SEQ ID NOs: 23-43 and 97-100. In some embodiments, a VHH domain that binds albumin comprises an amino acid sequence that is at least 85%, at least 90%, at least 95%, at least 96%, at least 97%, at least 98%, or at least 99% identical to SEQ ID NO: 99. In some embodiments, a VHH domain that binds albumin comprises an amino acid sequence of SEQ ID NO: 99.

In some embodiments, a VHH domain that binds albumin is provided, which competes for binding to albumin with a VHH domain comprising an amino acid sequence selected from SEQ ID NOs: 23-43 and 97-100.

### Exemplary FcRn- and Albumin-Binding Polypeptides

In some embodiments, polypeptides that comprise at least one VHH domain that binds FcRn and at least one VHH domain that binds albumin are provided. In various embodiments, the VHH domain that binds FcRn and/or the VHH domain that binds albumin is a VHH domain provided herein.

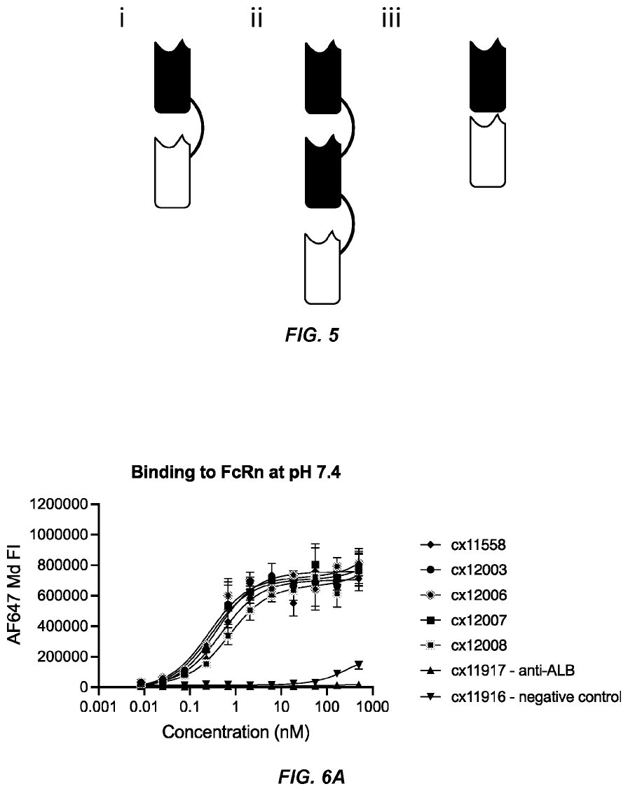

In some embodiments, a polypeptide comprising one, two, three, four, five, six, seven, or eight VHH domains that bind FcRn and at least one VHH domain that binds albumin is provided. In some embodiments, a polypeptide provided herein comprises one, two, three, or four VHH domains that bind FcRn and at least one VHH domain that binds albumin. Nonlimiting exemplary polypeptides comprising at least one VHH domain that binds FcRn and at least one FcRn that binds albumin are shown in FIG. 5.

In various embodiments, the polypeptides provided herein may comprise one or more additional antigen-binding domains that bind one or more target antigens other than albumin or FcRn. In various embodiments, a polypeptide that comprises one or more VHH domains that bind albumin and one or more VHH domains that bind FcRn also comprises a therapeutic antigen-binding domain and/or a therapeutic polypeptide. Such therapeutic antigen-binding domains include, but are not limited to, traditional antibody antigen-binding domains, which comprise a heavy chain variable region and a light chain variable region, and single-domain antibody antigen-binding domains, such as VHH domains. Nonlimiting exemplary therapeutic polypeptides include, for example, receptor extracellular domains, enzymes, and ligands. The VHH domain that binds FcRn and/or the VHH domain that binds albumin may be linked to the antigen-binding domain or therapeutic polypeptide at the N-terminus of the antigen-binding domain or therapeutic polypeptide, or at the C-terminus (e.g., VHH-VHH-antigen-binding domain or antigen-binding domain-VHH-VHH), or they may each be at different termini (e.g., VHH-antigen-binding domain-VHH).

In some embodiments of the polypeptides, adjacent portions of the polypeptide, such as a first VHH and a second VHH or a VHH and an antigen-binding domain, may be connected to one another by a linker. In some such embodiments, the linker comprises 1-20, 1-12, or 1-8 amino acids, preferably predominantly composed of glycine and, optionally, serine. A non-limiting example of such linker is GSGGGS (SEQ ID NO: 101). In some embodiments of the polypeptides, the C-terminal VHH may be extended by one or more additional amino acid residues. In certain embodiments such residues are glycine residues. In some embodiments, the C-terminal VHH is extended by two or more glycine residues.

In some embodiments, a polypeptide that comprises at least one VHH domain that binds albumin and one VHH domain that binds FcRn does not comprise an Fc region. In some embodiments, a polypeptide that comprises at least one VHH domain that binds albumin and one VHH domain that binds FcRn comprises an Fc region. In some embodiments, a polypeptide provided herein comprises one, two, three, or four VHH domains that bind albumin, one, two, three, or four VHH domains that bind FcRn and an Fc region. In some embodiments, an Fc region mediates dimerization of the polypeptide at physiological conditions.

In various embodiments, the polypeptide comprising at least one VHH domain that binds albumin and FcRn has a longer half-life in vivo than the same polypeptide without the at least one VHH domain that binds albumin. In some embodiments, the half-life is at least 1.5×, at least 2×, at least 3×, at least 4×, or at least 5× longer than the half-life of the polypeptide without the VHH domain that binds albumin.

In some embodiments, a polypeptide that comprises at least one VHH domain that binds albumin and one VHH domain that binds FcRn comprises a sequence at least 85%, at least 90%, at least 95%, at least 96%, at least 97%, at least 98%, or at least 99% identical to a sequence selected from SEQ ID NOs: 69-77. In some embodiments, a polypeptide that comprises at least one VHH domain that binds albumin and one VHH domain that binds FcRn comprises a sequence selected from SEQ ID NOs: 69-77.

In some embodiments, a polypeptide that comprises at least one VHH domain that binds albumin and one VHH domain that binds FcRn comprises a sequence at least 85%, at least 90%, at least 95%, at least 96%, at least 97%, at least 98%, or at least 99% identical to a sequence selected from SEQ ID NOs: 69-74, and 77. In some embodiments, a polypeptide that comprises at least one VHH domain that binds albumin and one VHH domain that binds FcRn comprises a sequence selected from SEQ ID NOs: 69-74, and 77.

### Humanized VHH Domains

In some embodiments, a VHH domain is humanized. Humanized VHH domains (such as in sdAbs or VHH-containing polypeptides) are useful as therapeutic molecules because humanized VHH domains reduce or eliminate the human immune response to non-human antibodies, which can result in an immune response to an antibody therapeutic, and decreased effectiveness of the therapeutic. Generally, a humanized VHH comprises CDRs (or portions thereof) derived from a non-human antibody and FRs (or portions thereof) derived from human antibody sequences. In some embodiments, some FR residues in a humanized VHH are substituted with corresponding residues from a non-human antibody (for example, the VHH from which the CDR residues are derived), for example, to restore or improve VHH specificity or affinity.

Humanized antibodies and methods of making them are reviewed, for example, in Almagro and Fransson, (2008) Front. Biosci. 13:1619-1633, and are further described, for example, in Riechmann et al., (1988) Nature 332:323-329; Queen et al., (1989) Proc. Natl Acad. Sci. USA 86:10029-10033; U.S. Pat. No. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272:10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

### Fc Domains

In various embodiments, an Fc region included in a polypeptide is a human Fc region, or is derived from a human Fc region.

In some embodiments, the Fc region included in a polypeptide is derived from a human Fc region and comprises mutations designed for heterodimerization, herein referred to as “knob” and “hole”. In some embodiments, the “knob” Fc region comprises the mutation T366W. In some embodiments, the “hole” Fc region comprises mutations T366S, L368A, and γ₄₀₇V. In some embodiments, Fc regions used for heterodimerization comprise additional mutations, such as the mutation S354C on a first member of a heterodimeric Fc pair that forms an asymmetric disulfide with a corresponding mutation γ₃₄₉C on the second member of a heterodimeric Fc pair. In some embodiments, one member of a heterodimeric Fc pair comprises the modification H435R or H435K to prevent protein A binding while maintaining FcRn binding. In some embodiments, one member of a heterodimeric Fc pair comprises the modification H435R or H435K, while the second member of the heterodimeric Fc pair is not modified at H435. In various embodiments, the hold Fc region comprises the modification H435R or H435K (referred to as “hole-R” in some instances when the modification is H435R), while the knob Fc region does not. In some instances, the hole-R mutation improves purification of the heterodimer over homodimeric hole Fc regions that may be present.

Nonlimiting exemplary Fc regions that may be used in a polypeptide include Fc regions comprising the amino acid sequences of SEQ ID NOs: 47-68.

### Polypeptide Expression and Production

Nucleic acid molecules comprising polynucleotides that encode a polypeptide provided herein are provided. In some embodiments, the nucleic acid molecule may also encode a leader sequence that directs secretion of the polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode a polypeptide provided herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In some embodiments, a polypeptide provided herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, the polypeptides may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a polypeptide described herein is provided. The polypeptides expressed in host cells can be purified by any suitable method. Such methods include, but are not limited to, the use of affinity matrices or hydrophobic interaction chromatography. Suitable affinity ligands include the RORI ECD and agents that bind Fc regions. For example, a Protein A, Protein G, Protein A/G, or an antibody affinity column may be used to bind the Fc region and to purify a polypeptide that comprises an Fc region. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

In some embodiments, the polypeptide is produced in a cell-free system. Nonlimiting exemplary cell-free systems are described, for example, in Sitaraman et al., Methods Mol. Biol. 498:229-44 (2009); Spirin, Trends Biotechnol. 22:538-45 (2004); Endo et al., Biotechnol. Adv. 21:695-713 (2003).

In some embodiments, a polypeptide prepared by the methods described above are provided. In some embodiments, the polypeptide is prepared in a host cell. In some embodiments, the polypeptide is prepared in a cell-free system. In some embodiments, the polypeptide is purified. In some embodiments, a cell culture media comprising a polypeptide is provided.

In some embodiments, compositions comprising antibodies prepared by the methods described above are provided. In some embodiments, the composition comprises a polypeptide provided herein prepared in a host cell. In some embodiments, the composition comprises a polypeptide prepared in a cell-free system. In some embodiments, the composition comprises a purified polypeptide.

### Exemplary Methods of Treating Diseases Using FcRn-Binding Polypeptides

In some embodiments, methods of treating disease in an individual are provided, comprising administering a therapeutic polypeptide comprising an FcRn-binding domain provided herein. Such diseases include any disease that would benefit from treatment with the therapeutic polypeptide comprising an FcRn-binding domain. Nonlimiting exemplary diseases that may be treated with therapeutic polypeptides comprising an FcRn-binding domain provided herein include infectious diseases, immunological diseases or disorders (e.g., autoimmune diseases or disorders), inflammatory diseases or disorders, and cancer. In some embodiments, the disease or disorder is an autoantibody-mediated disease or disorder. In some embodiments the disease or disorder is pemphigus vulgaris, lupus nephritis, myasthenia gravis, Guillain-Barré syndrome, antibody-mediated rejection, antiphospholipid antibody syndrome, chronic inflammatory demyelinating polyneuropathy, immune complex-mediated vasculitis, glomerulitis, a channelopathy, neuromyelitis optica, autoimmune encephalitis, autoimmune Grave's disease, idiopathic thrombocytopenia purpura (ITP), autoimmune haemolytic anaemia (AIHA), immune neutropenia, dilated cardiomyopathy, or serum sickness. The method comprises administering to the individual an effective amount of a therapeutic polypeptide comprising an FcRn-binding domain provided herein. In some embodiments, the method of treatment promotes the clearance of autoantibodies in a subject. In some embodiments, the method of treatment blocks an immune response, (e.g., block an immune complex-based activation of the immune response) in a subject. Such methods of treatment may be in humans or animals. In some embodiments, methods of treating humans are provided.

The therapeutic polypeptides comprising an FcRn-binding domain provided herein can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of a therapeutic polypeptide is administered to a subject one or more times. In some embodiments, an effective dose of a therapeutic polypeptide comprising an FcRn-binding domain is administered to the subject daily, semiweekly, weekly, every two weeks, once a month, etc. An effective dose of a therapeutic polypeptide comprising an FcRn-binding domain is administered to the subject at least once. In some embodiments, the effective dose of a therapeutic polypeptide may be administered multiple times, including multiple times over the course of at least a month, at least six months, or at least a year.

In some embodiments, pharmaceutical compositions are administered in an amount effective for treating disease. The therapeutically effective amount is typically dependent on the weight of the subject being treated, his or her physical or health condition, the extensiveness of the condition to be treated, or the age of the subject being treated.

In some embodiments, therapeutic polypeptides can be administered in vivo by various routes, including, but not limited to, intravenous, intra-arterial, parenteral, intraperitoneal or subcutaneous. The appropriate formulation and route of administration may be selected according to the intended application.

### Pharmaceutical Compositions

In some embodiments, compositions comprising polypeptides are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

### Nonlimiting Exemplary Methods of Diagnosis and Treatment

In some embodiments, the methods described herein are useful for evaluating a subject and/or a specimen from a subject (e.g. a patient suffering from an autoimmune disease or a cancer patient). In some embodiments, evaluation is one or more of diagnosis, prognosis, and/or response to treatment.

In some embodiments, the methods described herein comprise evaluating a presence, absence, or level of a protein. In some embodiments, the methods described herein comprise evaluating a presence, absence, or level of expression of a nucleic acid. The compositions described herein may be used for these measurements. In some embodiments, the evaluation may direct treatment (including treatment with the polypeptides described herein).

### Kits

Also provided are articles of manufacture and kits that include any of the polypeptides as described herein, and suitable packaging. In some embodiments, the invention includes a kit with (i) a polypeptide provided herein, and (ii) instructions for using the kit to administer the polypeptide to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of molecules disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of antibody.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Development of Anti-Albumin Single Domain Antibodies (sdAbs)

Single domain antibodies targeting human albumin were generated via immunization of llamas and/or alpacas with a recombinant version of human serum albumin (SEQ ID NO: 1).

Following the development of specific anti-albumin antibody titers, llama/alpaca peripheral blood mononuclear cells (PBMCs) were isolated from 500 mL of blood from the immunized animal and total mRNA was isolated using the Qiagen RNeasy Maxi Kit and subsequently converted to first strand cDNA using Thermo Superscript IV Reverse Transcriptase and oligo-dT priming. VHH sequences were specifically amplified via PCR using the cDNA as template and cloned into a yeast surface display vector as VHH-Fc-AGA2 fusion proteins. The Fc was a human IgG1 Fc or, in some cases, a variant IgG1 Fc with reduced effector function.

Yeast libraries displaying the VHH-Fc-AGA2 fusion proteins were enriched using recombinant forms of human albumin via magnetic bead isolation followed by fluorescence activated cell sorting (FACS). Sorted yeast were plated out and isolated colonies were picked into 96-well blocks and an induction of yeast cell surface expression of VHH-Fc-AGA2 fusion protein was conducted. Biotinylated recombinant human albumin or irrelevant biotinylated protein (albumin negative) were directly applied to induced yeast, washed, treated with fluorophore labelled streptavidin, and analyzed by 96-well flow cytometry.

Nucleic acid sequences encoding VHHs that bound to biotinylated recombinant human albumin and not to irrelevant biotinylated protein were cloned in-frame with a human Fc xELL encoding region into mammalian expression vectors, and expressed by transient transfection in HEK293 Freestyle cells (293F cells) or CHO cells using polyethylenimine. Supernatant was collected after 3-7 days, secreted recombinant protein was purified by protein A chromatography, and concentration was calculated from the absorbance at 280 nm and extinction coefficient.

Anti-albumin sdAb 4A01 was selected for humanization.

### Example 2: Humanization of Anti-Albumin sdAb 4A01 and Species Cross-Reactivity

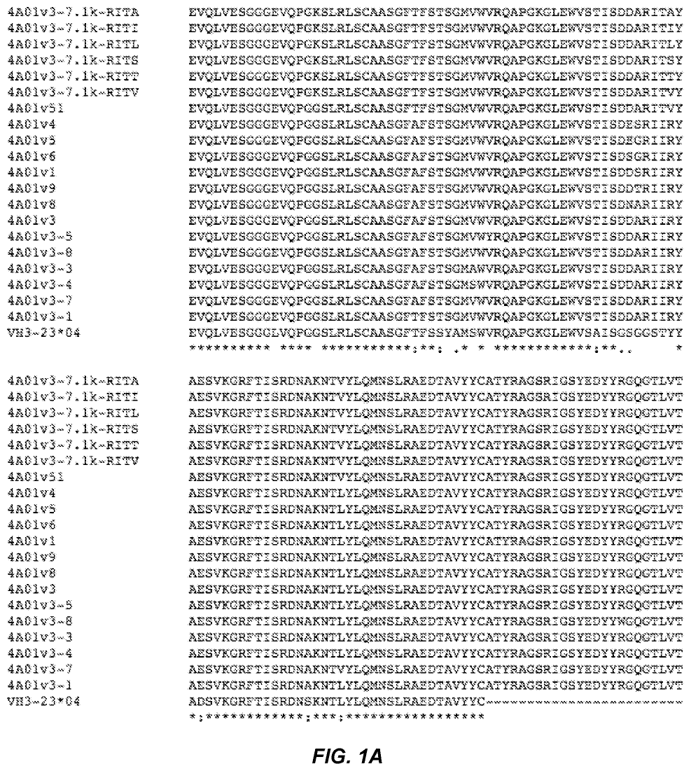

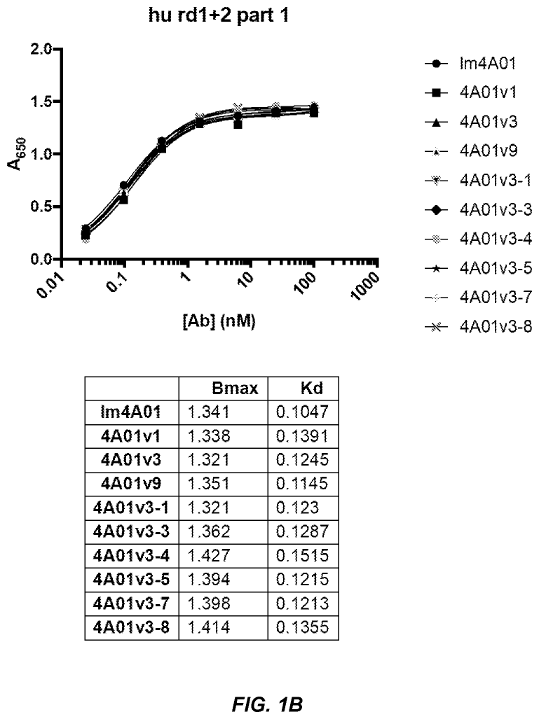

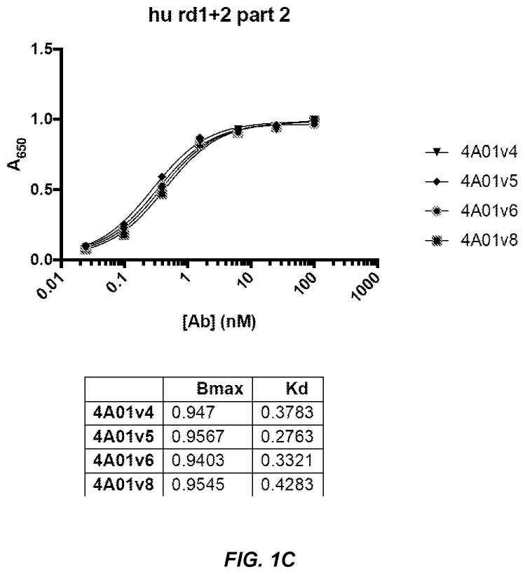

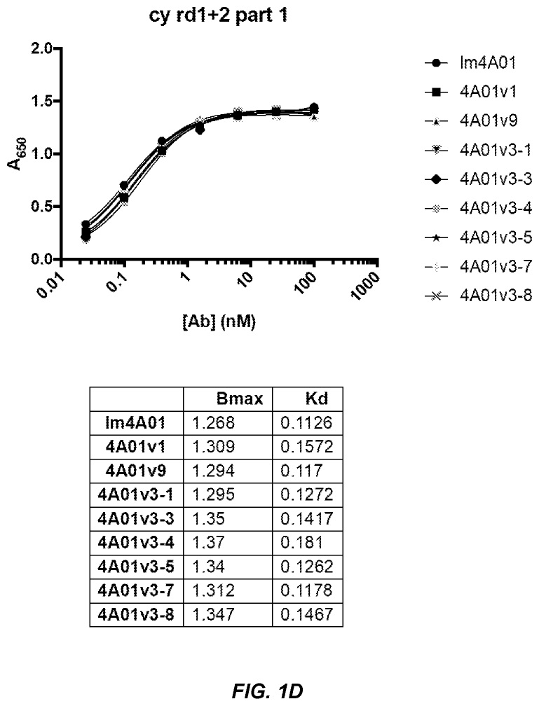

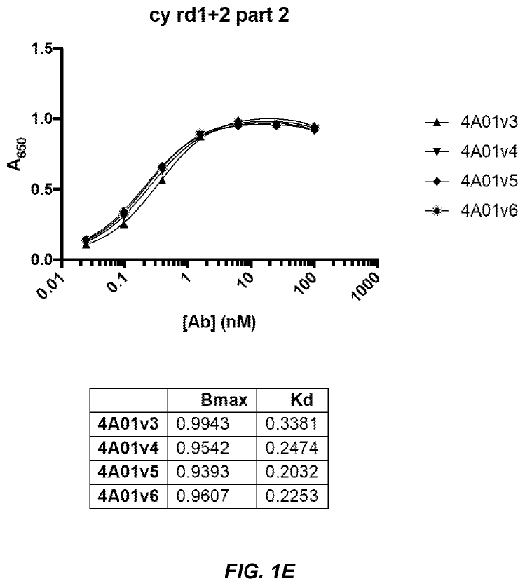

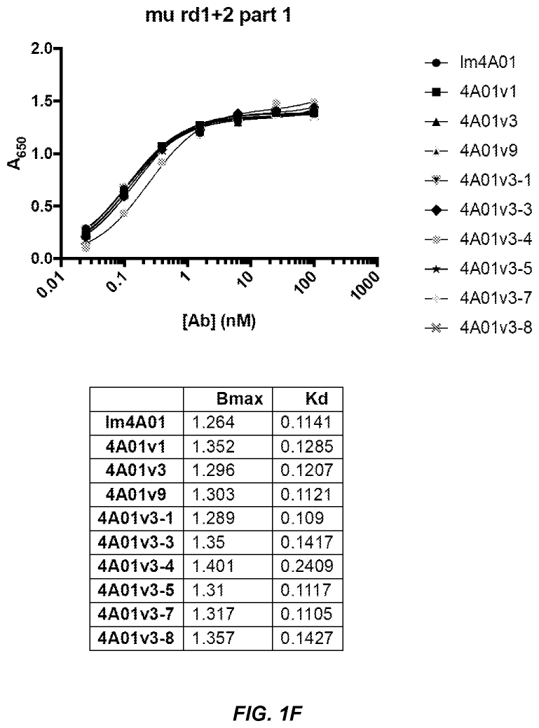

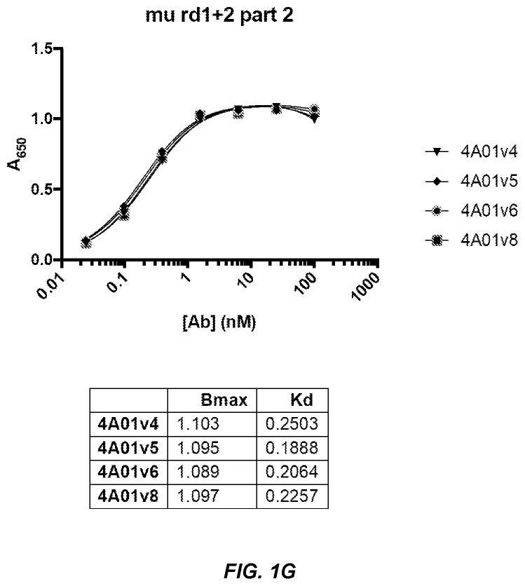

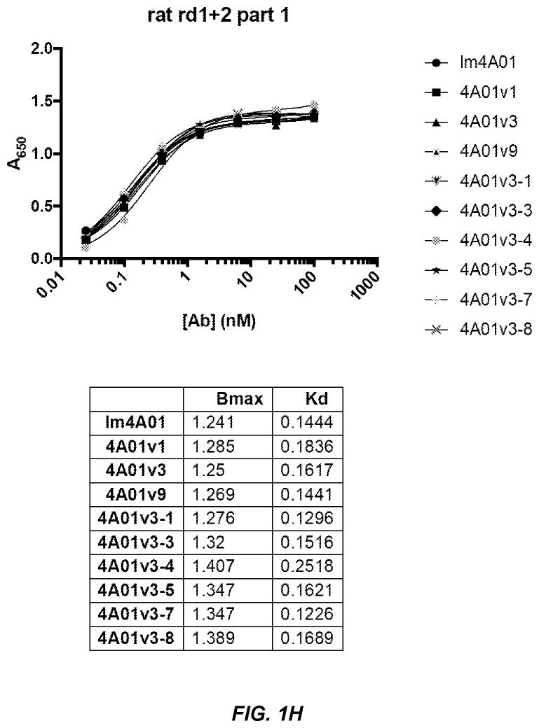

Various humanized forms of sdAb 4A01 were made based on the human heavy chain frameworks VH3-23*04. Certain amino acids were back-mutated to the donor amino acid, and certain mutations were tested, for example, in CDR2. FIG. 1A shows an alignment of the human heavy chain acceptor sequence with the humanized forms of 4A01.

Binding of monomeric anti-albumin sdAbs 4A01 (“1m4A01”) and its humanized versions to human serum albumin, cynomolgus serum albumin, murine serum albumin, and rat serum albumin was determined by ELISA as follows. Medisorp plates were coated with albumin protein at 2 μg/ml, 50 μl/well at 4° C. overnight (human, murine, and rat albumin-Sigma, cynomolgus monkey albumin-Abcam). 1× Fish Gelatin (blocking agent, Bethyl Laboratories) was added to albumin-coated wells followed by a 1 hour incubation at RT. Titrations of sdAb fusion proteins (starting at 100 nM, across 1:3 or down 1:4) were added and incubated for 1 hour at RT. Plates were washed 3 times with 0.1% D-PBST, and then anti-human Fc HRP antibody (1:2000 in 0.1% D-PBST, Jackson) was added and incubated for 30 minutes at RT. Plates were washed 3 times with 0.1% D-PBST, and then TMB substrate was added. Absorbance at 650 nm was read on a plate reader (Molecular Devices) and data were plotted using equation one site-total binding (Model: Y=Bmax*X/(Kd+X)+NS*X+Background, GraphPad Prism).

Binding of 4A01 and humanized forms of 4A01 to human albumin is shown in FIG. 1B-1C. All of the sdAbs bound human albumin with a K_{D} between 0.10 and 0.43 nM. Binding of 4A01 and humanized forms of 4A01 to cynomolgus monkey albumin is shown in FIG. 1D-1E. All of the sdAbs bound cynomolgus monkey albumin with a K_{D} between 0.11 and 0.34 nM. Binding of 4A01 and humanized forms of 4A01 to murine albumin is shown in FIG. 1F-1G. All of the sdAbs bound murine albumin with a K_{D} between 0.11 and about 0.25 nM. Binding of 4A01 and humanized forms of 4A01 to rat albumin is shown in FIG. 1H-1I. All of the sdAbs bound rat albumin with a K_{D} between 0.14 and about 0.33 nM.

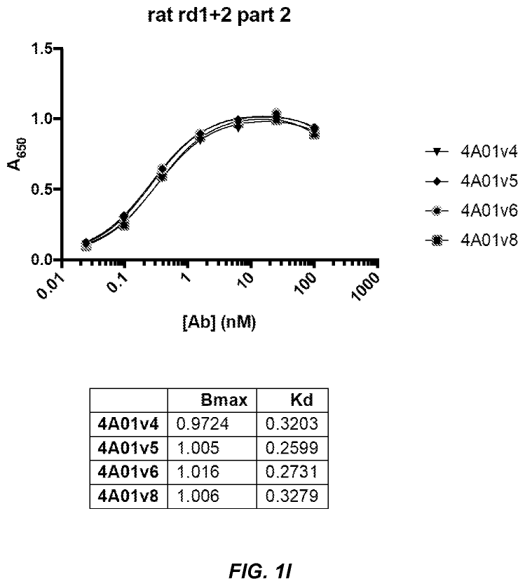

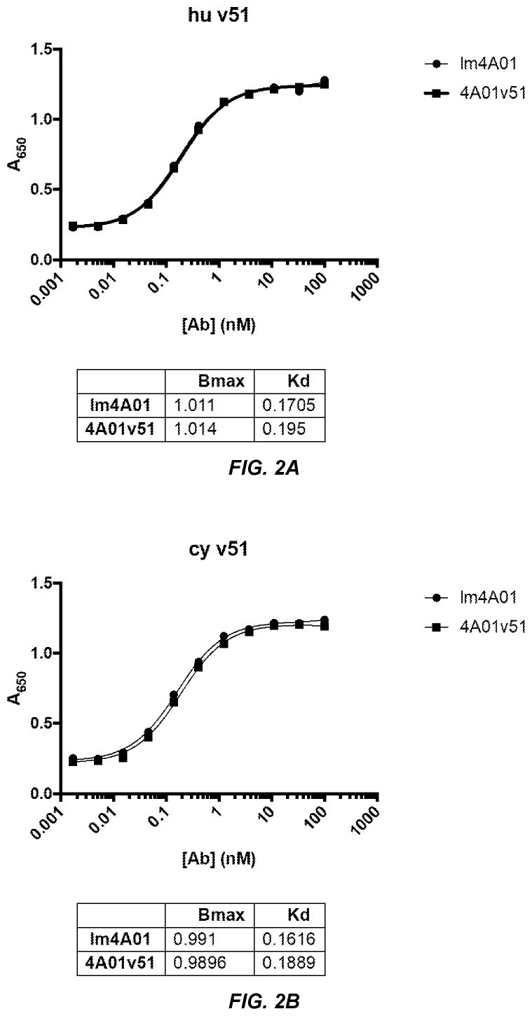

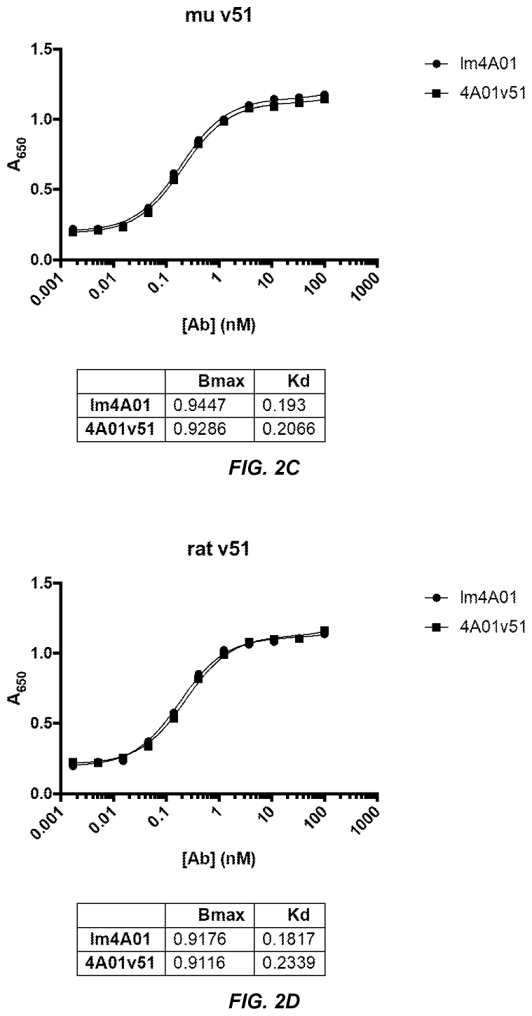

FIG. 2A-2D show binding of 4A01 and humanized hz4A01v51 to human (2A), cynomolgus monkey (2B), murine (2C), and rat (2D) albumin. 4A01 and all of the humanized variants tested bound all four species of albumin with an affinity of less than 1 nM. Humanized hz4A01v51 bound all four species of albumin with an affinity of less than 0.3 nM, and achieved maximal binding of greater than 90%.

The framework regions of hz4A01v51 were further modified, including by back-mutating certain residues to the donor amino acid and/or introducing alternative charged residues. Modified monomeric anti-albumin antibodies (comprising hz4A01v51.9, hz4A01v51.11, hz4A01v51.12, or hz4A01v51.13 VHH domains) exhibit similar binding profiles at pH 6 and pH 7.4 and all exhibited improved binding over that observed for hz4A01v51, particularly at pH 6 (data not shown).

### Example 3: Anti-Albumin sdAb 4A01 does not Bind Albumin Domain 3

Albumin binds to the beta-2 microglobulin FcRn complex primarily through domain 3, and that binding is believed to be involved in the improved half-life of proteins fused to anti-albumin antibodies, or fused to albumin itself. To determine whether anti-albumin sdAb 4A01 binds to albumin domain 3, binding of 4A01-NNT-hFc was assayed by biolayer interferometry, as follows.

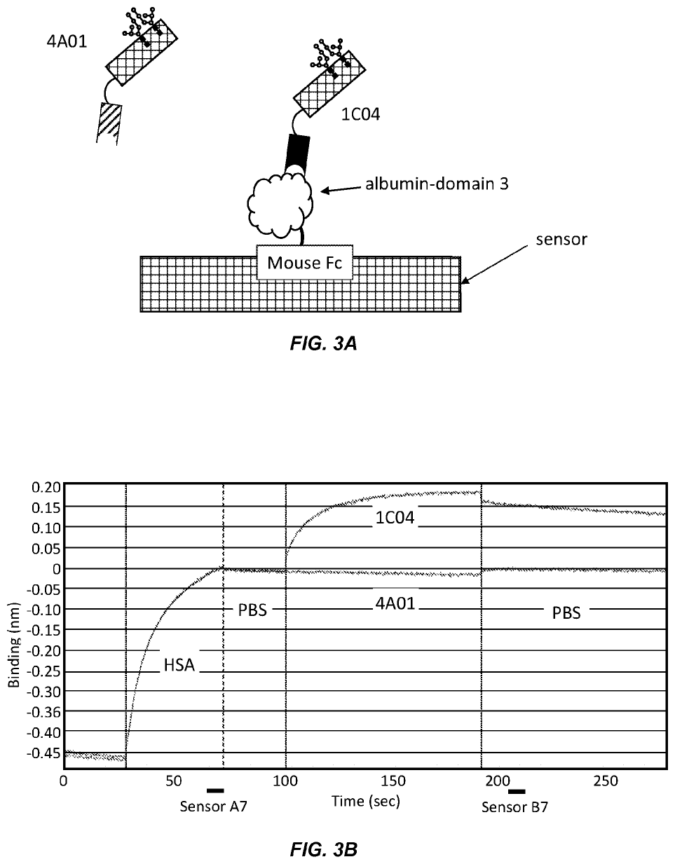

Albumin domain 3 (mouse Fc tagged) was immobilized on anti-mouse IgG Fc capture biosensor. All buffers/protein formulations were in MBST5 (50 nM MES pH5, 150 mM NaCl, 0.025% Tween)). A baseline was established with buffer only. Mouse Fc-tagged human albumin domain III (10 μg/ml) was loaded onto the anti-mouse IgG Fc capture biosensors (ForteBio). Anti-albumin sdAbs 4A01 (4A01-NNT-hFc) and 1C04 (similar format) were then loaded and allowed to associate with the captured biotin domain 3, followed by dissociation with MBST5. sdAb 1C04 is known to bind to albumin domain 3, and was used as a positive control. See FIG. 3A.

As shown in FIG. 3B, 1C04, but not 4A01, bound to the immobilized albumin domain 3.

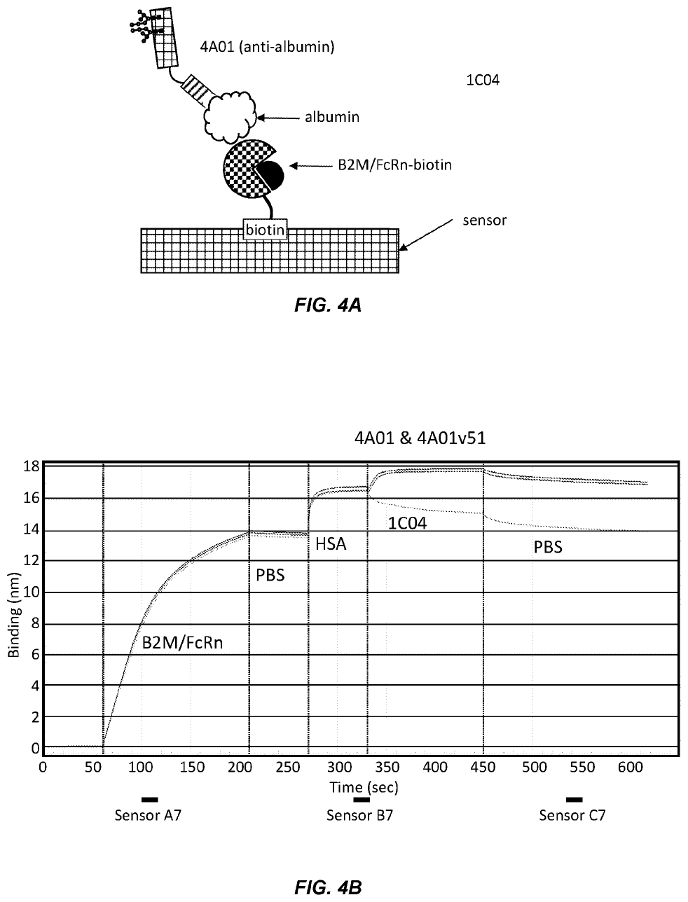

Anti-albumin sdAb 4A01 (4A01-NNT-hFc), hz4A01v51, and 1C04 were then tested for interference with albumin-FcRn binding, as follows. Binding was assessed by biolayer interferometry using biotinylated recombinant FcRn-B2M immobilized on a streptavidin biosensor. The immobilized FcRn-B2M was then complexed with recombinant human albumin. All buffers/protein formulations were in MBST5 (50 mM MES pH5, 150 mM NaCl, 0.025% Tween). A baseline was established with buffer only. Biotinylated FcRn-B2M (10 μg/ml, Acro Biosystems) was loaded onto the streptavidin biosensors (ForteBio), and a further baseline determined. 50 μM recombinant human albumin (Sigma) was then added and allowed to associate with the immobilized FcRn-B2M. Anti-albumin sdAbs 4A01 and 4A01v51, and sdAb 1C04 were then loaded and allowed to associate with the captured biotin domain 3, followed by dissociation with MBST5. See FIG. 4A

As shown in FIG. 3B, 4A01 and hx4A01v51 both bound to albumin associated with FcRn-B2M, but 1C04 did not.

### Example 4: Development of FcRn Single Domain Antibodies (sdAbs)

Single domain antibodies targeting human FcRn were generated via immunization of llamas with a polypeptide comprising human B2m (SEQ ID NO: 79) and the extracellular domain of human FcRn (SEQ ID NO: 78) joined with a flexible linker and fused to a polyhistidine tag for purification. Following the development of specific anti-FcRn antibody titers, llama peripheral blood mononuclear cells (PBMCs) were isolated from 500 mL of blood from the immunized animal and total mRNA was isolated using the Qiagen RNeasy Maxi Kit and subsequently converted to first strand cDNA using Thermo Superscript IV Reverse Transcriptase and oligo-dT priming. VHH sequences were specifically amplified via PCR using the cDNA as template and cloned into a yeast surface display vector as VHH-Fc-AGA2 fusion proteins.

Yeast libraries displaying the VHH-Fc-AGA2 fusion proteins were enriched using recombinant forms of the FcRn ECD via magnetic bead isolation followed by fluorescence activated cell sorting (FACS). Sorted yeast were plated out and isolated colonies were picked into 96-well blocks and grown in media that switched the expression from surface displayed VHH-Fc to secretion into the media. Supernatants from the 96-well yeast secretion cultures were applied to 293F cells transiently transfected with FcRn (FcRn positive) or untransfected 293F cells (FcRn negative), washed, treated with fluorophore labelled anti-human IgG1 Fc secondary antibody, and analyzed by 96-well flow cytometry.

One VHH domain that binds FcRn (clone B10a) was humanized substantially as described in Example 2. Humanized B10a VHH domains (hzB10a.v1-v2.5) were used to generate bispecific polypeptides targeting human FcRn and albumin having the structure shown in FIG. 5(i) (cx11383, cx11558, cx12003, cx12006, cx12007, cx12008, and cx12032) or FIG. 5(ii) (cx11385 and cx11642).

### Example 5: Binding of Anti-FcRn VHH Polypeptides to Human FcRn

Binding of anti-FcRn VHH polypeptides to human FcRn at neutral (7.4) or endosomal (6) pH was tested by flow cytometry using a CHO-based overexpression system or isolated human peripheral blood mononuclear cells (PBMC). CHO cells were transiently transfected to overexpress human FcRn extracellular domain (ECD) (SEQ ID NO: 78) fused to a transmembrane domain and an intracellular citrine and Beta-2 microglobulin (SEQ ID NO: 79).

CHO cells were plated in 96-well plates at 50,000 cells per well and resuspended in PBS, pH 7.4 or a buffer containing 20 mM His, 150 mM NaCl, pH 6. Serial dilutions of test articles were prepared at 2× the final concentration in the buffers described above (pH 7.4 or pH 6) and added to the cell suspensions. Plates were incubated for 60 minutes at 4° C. After the incubation cells were washed in the respective buffer and then incubated for 30 minutes at 4° C. with a fluorescently labeled (AF647) antibody detecting the VHH polypeptides. Binding of the detection antibody to FcRn on transfected cells (citrine positive) was measured after a final wash using an iQue Screener (Sartorius).

Isolated human PBMC were plated in 96-well plates at 120,000 cells per well and washed with PBS, pH 7.4. Cells were stained with Zombie Aqua to exclude dead cells and with specific fluorescently labeled antibodies against surface lineage markers (CD14, CD16, CD3, CD56, CD19 and HLA-DR) to allow gating on different PBMC subpopulations. After the surface marker staining, cells were fixed and permeabilized using a Foxp3/Transcription Factor Fixation/Permeabilization kit (eBioscience™) according to the manufacturer's recommended protocol. Cells were then resuspended in the buffers described above to generate pH 7.4 or pH 6 conditions and test article staining was carried out as described for CHO cells. After the final wash, binding to FcRn on PBMC subpopulations (Monocytes, B cells or T cells) was measured on a Novocyte-Quanteon Flow Cytometer (Agilent).

Binding of some anti-FcRn VHH polypeptides to human FcRn at neutral (7.4) pH was also tested by ELISA. 96-well ELISA plates were coated with human FcRn/B2m heterodimer protein in PBS overnight at 4° C., washed with PBST and then blocked with 3% BSA in PBST for 1 h at RT. Serial dilutions of test articles were prepared in PBST pH 7.4 and added to the plates. Plates were incubated for 1 h at room temperature. After the incubation cells were washed in the PBST and then incubated for 1 h at room temperature with a horse radish peroxidase (HRP)-conjugated antibody detecting the polypeptides. Plates were washed with PBS-T followed by addition of TMB substrate and color allowed to develop. TMB substrate was stopped with an equal volume of stop solution (1M H₂SO₄). Absorbance at 450 nm was measured with a 96 well plate reader (Emax, Molecular Devices). All data were plotted and analyzed using GraphPad Prism analysis software.

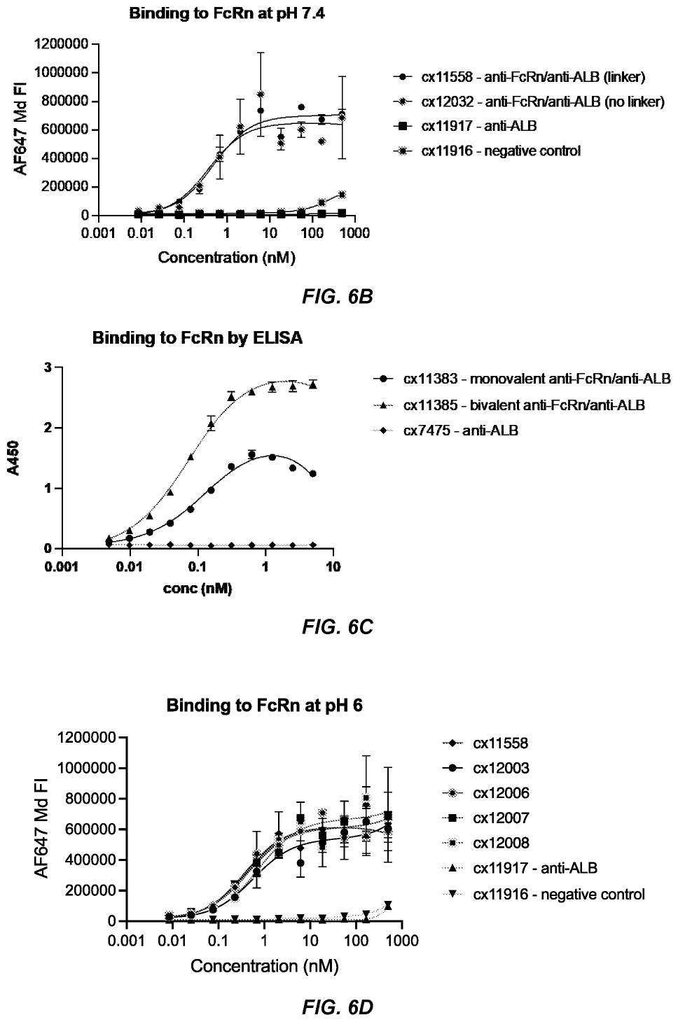

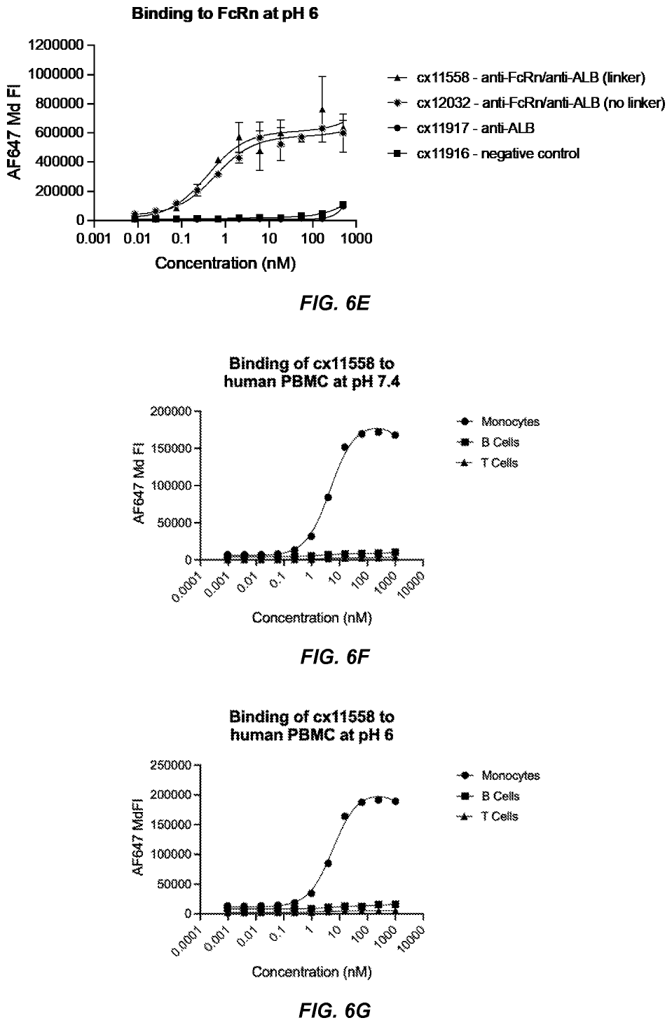

As shown in FIGS. 6A-B and 6D-E, all tested FcRn-targeting polypeptides bind to human FcRn overexpressed on CHO cells with low to sub-nanomolar apparent affinity. The binding is independent of the pH and comparable at pH 7.4 and pH 6. As shown in FIG. 6C, polypeptides with bivalent FcRn binding domains (as shown in FIG. 5(ii)) bind to FcRn with higher affinity than their monovalent counterparts (as shown in FIG. 5(i)). Binding is mediated by the VHH units specific to FcRn, as a polypeptide formatted as shown in FIG. 5(i) with monovalent specificity to albumin and a non-mammalian targeted VHH unit does not bind to human FcRn with appreciable affinity. FIGS. 6F and 6G show that pH-independent binding to FcRn is preserved on primary human cells. A polypeptide targeting FcRn and albumin (cx11558) formatted as shown in FIG. 5(i) binds FcRn-expressing human monocytes with low nanomolar affinity at pH 7.4 and pH 6 but does not bind to FcRn negative B or T cells.

### Example 6: Binding of Polypeptides to Human Albumin

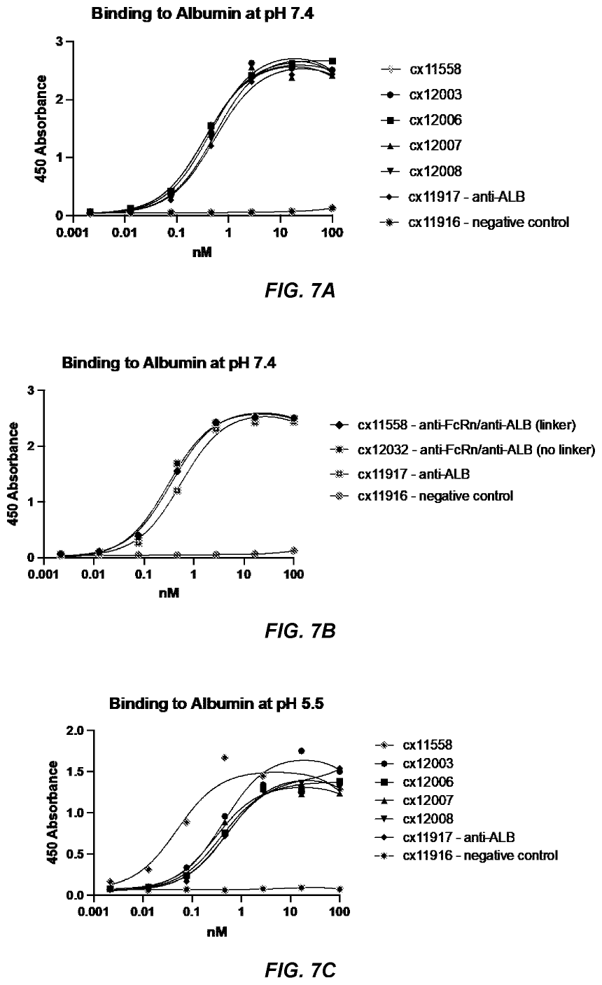

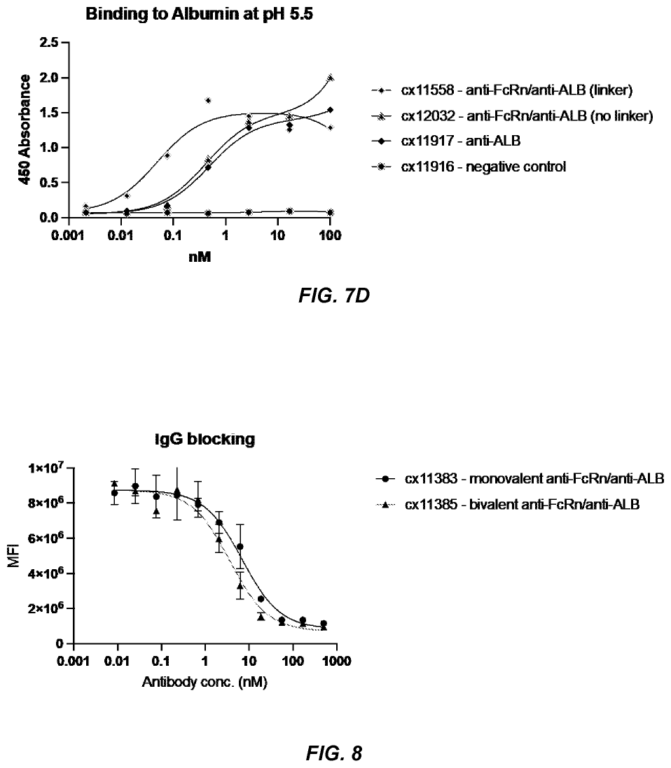

Binding of humanized VHH-containing polypeptides to human albumin at neutral (7.4) or late endosomal (5.5) pH was tested by ELISA. 96-well ELISA plates were coated with albumin protein in PBS overnight at 4° C., washed with PBS-T and then blocked with 5% milk powder in PBS-T for 1 h at room temperature. Serial dilutions of test articles were prepared in PBS pH 7.4 or a buffer containing 20 mM His, 150 mM NaCl, pH 5.5 and added to the plates. Plates were incubated for 1 h at room temperature. After the incubation cells were washed in the respective buffer and then incubated for 30 min at room temperature with a HRP-conjugated antibody detecting the polypeptides. Plates were washed in their respective buffers and TMB substrate was added and color allowed to develop. TMB substrate was stopped with an equal volume of stop solution (1M H₂SO₄). Absorbance at 450 nm was measured with a 96 well plate reader. The data were plotted and analyzed using GraphPad Prism analysis software. The results are shown in FIG. 7A-7D.

As shown in FIG. 7A-7D, all tested bi-specific anti-FcRn/anti-Albumin polypeptides bind to human albumin with low to sub-nanomolar apparent affinity. The binding is independent of the pH and comparable at pH 7.4 and pH 5.5. Binding is mediated by the VHH units specific to Albumin, as a polypeptide formatted as shown in FIG. 5(i) with monovalent specificity to albumin and a non-mammalian targeted VHH unit does not bind to human albumin with appreciable affinity.

### Example 7: Blocking of IgG Binding to Human FcRn by VHH-Containing Polypeptides

Blocking of human IgG binding to human FcRn expressed on HEK 293 cells by humanized VHH-containing polypeptides targeting human FcRn was measured by flow cytometry. HEK 293 cells were transiently transfected to overexpress human FcRn extracellular domain (ECD) (SEQ ID NO: 78) fused to a transmembrane domain and an intracellular citrine and Beta-2 microglobulin (SEQ ID NO: 79). 50,000 transiently transfected HEK 293 cells were plated in 96-well plates and resuspended in 20 mM His, 150 mM NaCl, pH 6. Serial dilutions of test articles were prepared at 2× the final concentration in the buffer described above and added to the cell suspensions. Plates were incubated for 60 minutes at 4° C. After the incubation biotinylated human IgG was added at a final concentration of 1 μM and incubated for an additional 60 min. Cells were then washed in the buffer described above and subsequently incubated for 30 minutes at 4° C. with a fluorescently labeled (AF647) streptavidin to detect IgG binding to FcRn. Binding of streptavidin to cells expressing FcRn (citrine positive) was measured after a final wash using an iQue Screener (Sartorius). The data were plotted and analyzed using GraphPad Prism analysis software. The results are shown in FIG. 8.

As shown in FIG. 8, increasing concentrations of the polypeptide test articles reduce the binding of human IgG to FcRn expressed on HEK 293 cells as measured by a reduction of mean fluorescence intensity (MFI) of the AF647-conjugated streptavidin reagent used to detect the bound biotinylated human IgG. The inhibitory activity (IC50) is similar between the construct formatted as described in FIG. 5(i) (cx11383) and the construct formatted as described in FIG. 5(ii) (cx11385) and falls in the low nano-molar range for both test articles.

### Example 8: In Vivo Pharmacokinetic Profile and IgG Depletion of Polypeptides Targeting Human FcRn and Albumin

The pharmacokinetic (PK) profile of humanized polypeptides targeting human FcRn and albumin was tested in regular BALB/c mice and transgenic animals expressing human FcRn and human albumin on a C57BL/6 background. The FcRn targeted VHH sequences used are not cross-reactive to mouse FcRn, but the albumin targeting VHH sequences bind mouse albumin (data not shown). Therefore, the ability to deplete IgG in vivo was assayed only in the human FcRn/albumin transgenic mouse system after injection of human IgG.

For the assessment of serum exposure in BALB/c mice, animals were injected intravenously with either 30 mg/kg or 0.3 mg/kg single doses of polypeptide test articles. Serum samples were drawn 30 min, 6 h, 24 h, 96 h and 168 h after the test article injection. Human FcRn/albumin transgenic mice were first injected with 500 mg/kg human IgG and then 24 h later with 20 mg/kg single doses of polypeptide test articles. Serum samples were drawn for these mice 30 min, 24 h, 96 h and 168 h after the test article administration. Test article concentrations and levels of human IgG in mouse serum were determined by ELISA. For the test article PK ELISA, human FcRn/B2M heterodimeric protein (His-tag, Acro Biosystem) was immobilized on 96-well ELISA plates by incubating 4 μg/mL of a protein solution in PBS for 12 h at 4° C. The next day plates were blocked with a 3% BSA TBS-T buffer for 2 h before incubation of the serum samples on these plates for 2h. Binding of test article in the serum samples to the FcRn immobilized on the ELISA plates was detected using a horse radish peroxidase (HRP)-conjugated secondary detection antibody able to bind the polypeptide. The secondary antibody was incubated on the plates for 1 h and binding was visualized using a TMB substrate solution followed by addition of stop solution (1<H₂SO₄) and measuring the absorbance at 450 nm on an Emax spectrophotometer (Molecular Devices).

Human IgG levels in mouse serum were determined by ELISA as well. A polyclonal goat capture antibody specific to human IgG was coated on 96-well ELISA plates at 1 μg/mL in PBS for 12 h at 4° C. The next day plates were blocked with a 1% BSA in PBS buffer for 2 h before incubation of the serum samples on these plates for 2h. Binding of human IgG in the serum was detected using a second goat anti-human IgG horse radish peroxidase (HRP)-conjugated detection antibody able to bind IgG in addition to the capture antibody. The secondary antibody was incubated on the plates for 1 h and binding was visualized as described above. Absorbance values measured for each ELISA were converted into test article concentrations in SoftMax Pro using standard curves from proteins with known concentrations analyzed in parallel with each assay. 4-parameter logistic regression was used to fit the standard curve. Data were exported and graphed using GraphPad Prism analysis software.

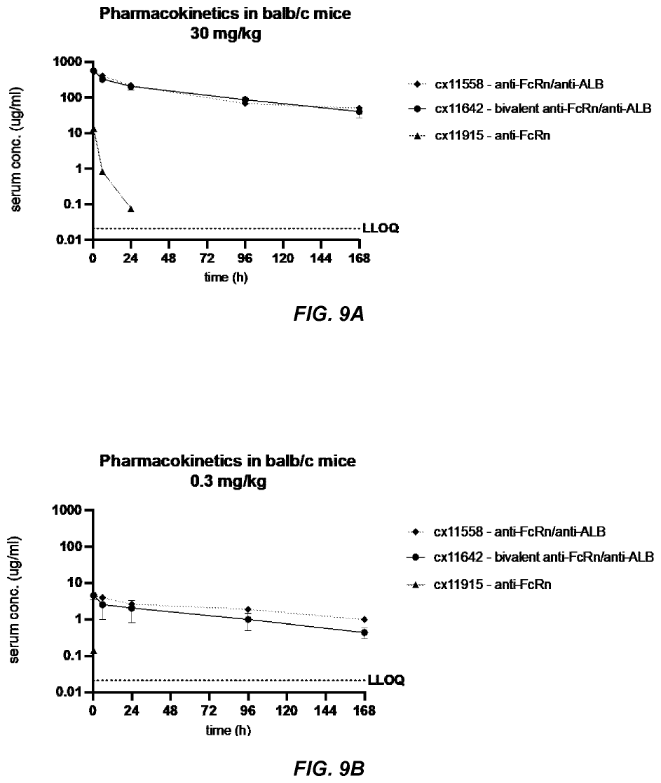

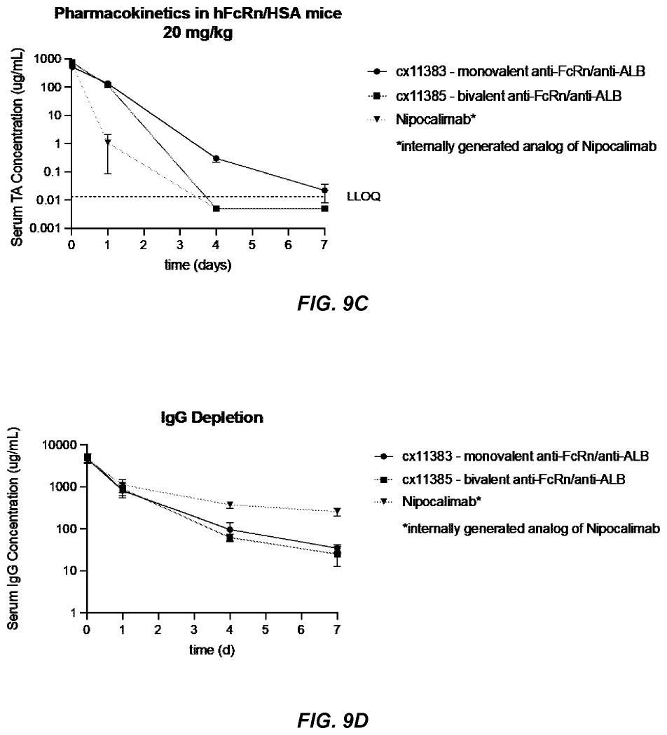

As shown in FIGS. 9A and 9B, the serum cMax levels at 30 min and the overall serum exposure of multi-valent, multi-specific polypeptides targeting FcRn and albumin formatted as described in FIG. 5(i) (cx11558) and FIG. 5(ii) (cx11642) are significantly higher than that of a polypeptide without albumin binding (cx11915). Both bi-specific formats show a similar PK profile at the dose levels tested. FIG. 9C shows the PK profile of two similar constructs formatted as described in FIG. 5(i) (cx11383) and FIG. 5(ii) (cx11385) and highlights their serum exposure compared to a Nipocalimab (anti-FcRn antibody) sequence analog generated based on publicly available information. All three test articles start at similar cMax serum concentrations. However, the time of exposure is significantly longer for the multi-valent, multi-specific single-polypeptides compared to the conventional antibody Nipocalimab. Between the two polypeptides, the construct formatted as described in FIG. 5(i) (cx11383) has a longer serum half-life. The expanded serum exposure also translates into better depletion of pre-injected human IgG in the human FcRn/albumin transgenic mouse system. Complexes cx11383 and cx11385 are both inducing accelerated clearance of human IgG compared to the Nipocalimab sequence analog and result in lower remaining serum IgG levels at the study terminus (168h). See FIG. 9D.

### Example 9: In Vivo IgG Depletion Mediated by Polypeptides Targeting Human FcRn and Albumin in Cynomolgus Monkeys

The pharmacodynamics (PD) and pharmacokinetics (PK) profile of humanized polypeptides targeting human FcRn and albumin were evaluated in several studies in in cynomolgus monkeys. In one study, naïve male cynomolgus monkeys 4-5 years of age were split into 5 treatment groups containing 2 animals per group. Groups 1 and 2 were treated with 100 mg/kg and 20 mg/kg cx11558 (monovalent) respectively, groups 3 and 4 were treated with 100 mg/kg and 20 mg/kg cx11642 (bivalent for FcRn binding) respectively, and group 5 was treated with 20 mg/kg efgartigimod (NDC 73475-3041-5, Lot #AHUC01A, a recombination engineered IgG Fc fragment). All treatments were administered via I.V. with a 60 min infusion at 10 mL/kg. Serum samples were collected from approximately 2 mL of whole blood from all animals and all groups pre-dose, at the end of infusion, and 24, 48, 72, 96, 120, 144, 168, 192, 216, 240, 264, 288, 312, 336, 384, 432, 480, 552, 600, 648, 696, 744, 792, and 840 hours post-dose.

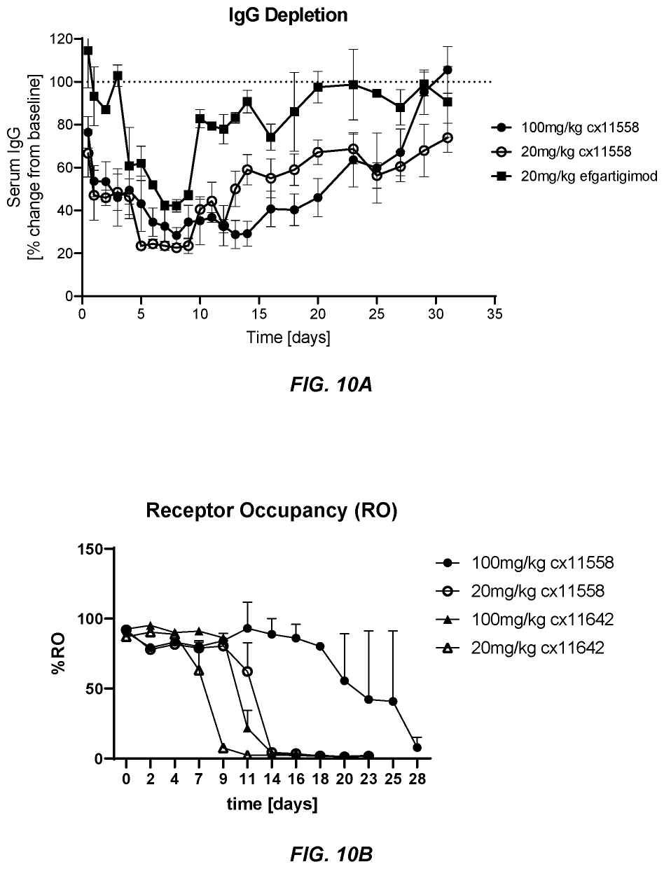

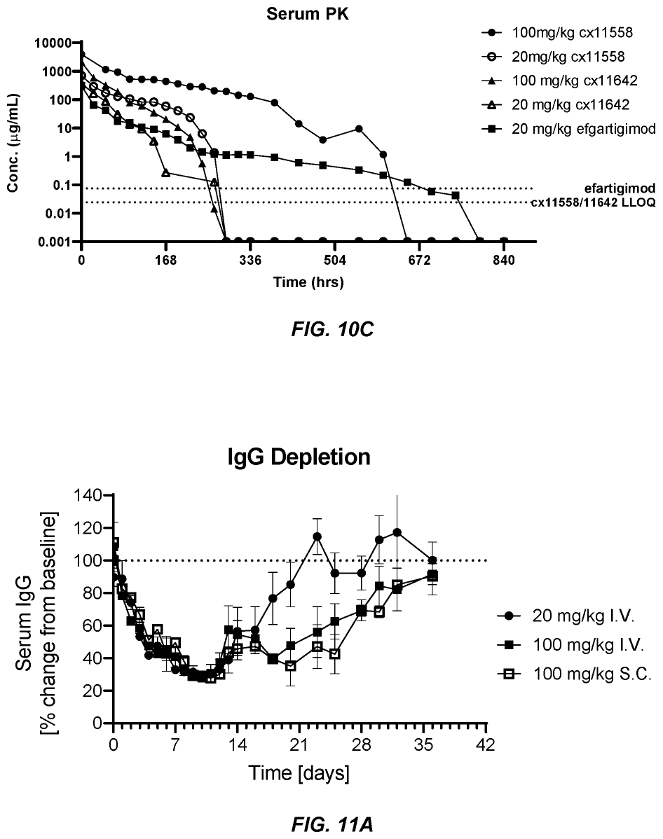

Cynomolgus IgG concentration was analyzed via a Monkey Cynomolgus IgG ELISA kit from Molecular Innovations (IMNCYIGGKT) per the manufacturer's protocol. Briefly, the Cynomolgus IgG standard dilutions were added to pre-coated ELISA plates in duplicate and serum samples prepared at an end dilution of 2×10\hat{ }6-3×10\hat{ }6 were added in triplicate and plates were incubated for 30 minutes. Plates were then washed and incubated with biotinylated anti-cyno monkey IgG primary antibody for 30 minutes, washed and incubated with horseradish peroxidase-conjugated streptavidin antibody for 30 minutes. Plates were then washed and TMB substrate solution was added to all wells for 2-10 minutes, the reaction was quenched via addition of 1N H₂SO₄ to all wells. Absorbance at 450 nM was measured using a plate spectrophotometer, background was subtracted from all standards and samples, the A450 was plotted against the amount of IgG in all standards, and the standard curve was analyzed using a four parameter logistic (4PL) curve fit in prism. The amount of IgG in all samples was determined using the standard curve and this value was multiplied by the dilution factor used for each serum sample. The IgG concentration (as percent change in from baseline) over time for the cx11558 and efgartigimod treatment groups is plotted in FIG. 10A.

FcRn occupancy by cx11558 and cx11642 on monocytes was examined by flow cytometry from whole blood samples. Whole blood was treated with RBS lysis buffer, centrifuged. The cell pellet was washed, resuspended in PBS, and split into 3 separate wells for staining. All cells were stained with ZombieAqua (Biolegend), incubated with cyno IgG (diluted to 0.2 mg/mL) in FACS buffer for 10 minutes, and the following antibody panel: CD14-BV421; CD16-BV605; CD8-BV785; CD20-PE/Cy7; NKG2A-PE; CD3-PE-Dazzle 594; HLA-DR-APC-Cy7 (sourced from Biolegend or BD), was added and incubated for an additional 30 minutes at 4° C. in FACS buffer. After washing, the cells were treated with eBioscience fix/perm for 30 minutes at 4° C., washed twice with cold perm buffer, and then incubated with saturating concentrations of B10a.v2.4 VHH-Avl-His (in house reagent, to detect free FcRn), saturating concentrations of unlabeled test article (cx11558 or cx11642; to detect total FcRn), or buffer (to detect occupied FcRn) for 30 minutes at 4° C. Cells were then washed, and incubated with streptavidin-AF647 (to detect B10av2.4VHH-Avi-His) or anti-id-AF647 (in house reagent, to detect cx11558 and cx11642) for 30 minutes at 4° C. After washing cells were resuspended in FACS buffer and analyzed using the Novocyte Quanteon flow cytometer. FcRn occupancy by cx11558 and cx11642 are shown in FIG. 10B.

The serum levels of cx11558, cx11642, and efgartigimod over time was determined by ELISA. Biotinylated human FcRn was coated to pre-blocked wells of a Pierce™ Streptavidin Coated High Capacity Plate, and standard, controls, and serum samples were diluted and added to the plate in duplicate. Following a 90-120 minute incubation, the plates were washed and incubated with either an anti-id-HRP conjugate (in house reagent for detection of cx11558 or cx11642) or a Mouse Anti-Human IgG Fc HRP conjugate (for detection of efgartigimod) for 60 minutes. Plates were then washed and a TMB substrate was added to all wells for 2-10 minutes, the reaction was quenched via addition of IN H₂SO₄ to all wells, and the absorbance is measured at 450 nM background (650 nM) was subtracted from all standards and samples on a SpectraMax M5e plate reader and reported in OD450-650. Absorbance and concentration of the standards are plotted and a curve fit using a 4PL regression in Softmax Pro v.5.4.1 or later. Sample concentrations are then interpolated from the standard curve regression and reported in ng/ml mass units. The serum concentration over time is plotted in FIG. 10C.

As shown in FIG. 10A cx11558 administered at either dose induced accelerated clearance of cynomolgus IgG compared to the efgartigimod and result in lower remaining serum IgG levels through at least day 27. Cx11642 administered at either dose also induced accelerated clearance of cynomolgus IgG below the levels observed with efgartigimod however, serum IgG levels appeared to return to normal levels faster than observed with cx11558 at the 100 mg/kg matched dose (data not shown). As shown in FIG. 10B cx11558 administered at 20 mg/kg and 100 mg/kg maintained full FcRn occupancy on monocytes for about 9 days and about 18 days, respectively, while cx11642 maintained full FcRn occupancy for about half as long. As shown in FIG. 10C the serum concentration of cx11558 administered at 20 mg/kg and 100 mg/kg doses remains above 10 μg/mL (which correlates with ˜100% receptor occupancy) for about 9 days and about 18 days, respectively, while the serum concentration of cx11642 at 20 mg/kg and 100 mg falls below 10 μg/mL by day 6 and 8, respectively and efgartigimod falls below 10 μg/mL by day 6.

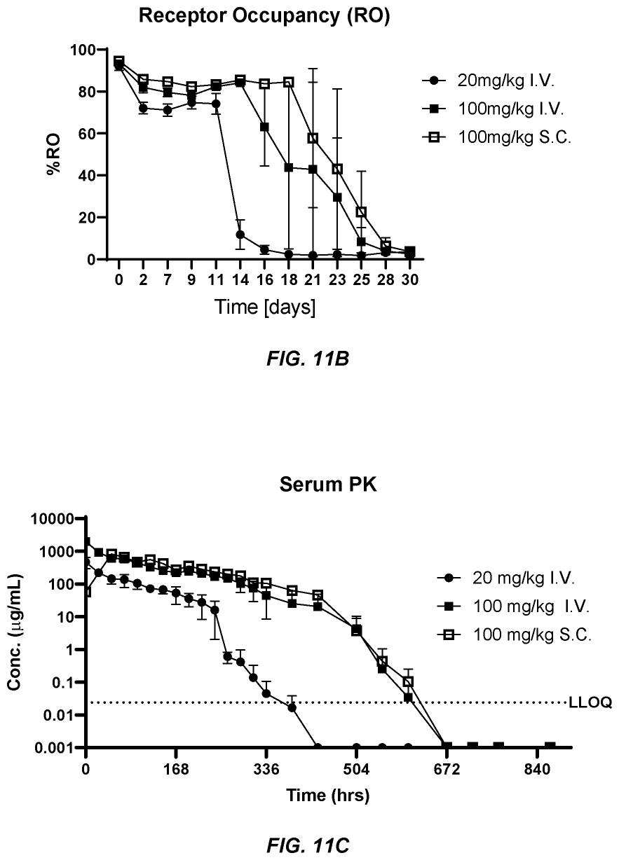

In a further study, the PD and PK profiles of a humanized polypeptide targeting human FcRn and albumin administered by intravenous infusion (I.V.) or subcutaneous injection (S.C.) were evaluated. For this study naïve male cynomolgus monkeys were split into 3 treatment groups containing 2 animals per group. Groups 1 and 2 were treated with a single dose of 20 mg/kg and 100 mg/kg of cx12007 respectively administered I.V. (70 min infusion at 10 mL/kg), group 3 was treated with a single dose of 100 mg/kg cx12007 administered S.C. (1 mL/kg). Serum samples were collected from whole blood from all animals and all groups pre-dose, at the end of infusion, and 24, 48, 72, 96, 120, 144, 168, 192, 216, 240, 264, 288, 312, 336, 384, 432, 504, 552, 600, 648, 696, 744, 792, and 840 hours post-dose. Cynomolgus IgG concentration was analyzed via a NHP Isotyping Kit from Meso Scale Discovery (K15203D) per the manufacturer's protocol. Briefly, the calibrator standards and serum samples prepared at an end dilution of 2×10\hat{ }5 were added to the MSD plates in duplicate and plates were incubated for 2 hours. Plates were then washed and incubated in detection antibody solution for 2 hours. After an additional wash, read buffer was added and the plates were immediately read on the MSD imager. The standard curve was analyzed using a 4 parameter logistic curve fitting model with a 1/Y² weighting function of the calibrator values. The amount of IgG in all samples was determined using the standard curve and this value was multiplied by the dilution factor used for each serum sample. The IgG concentration (as percent change in from baseline) over time for each of the treatment groups is plotted in FIG. 11A. FcRn occupancy on monocytes was examined by flow cytometry essentially as described above except saturating concentrations of unlabeled cx12007 (to detect total FcRn) were used. FcRn occupancy by cx12007 is shown in FIG. 11B. The serum concentration of cx12007 was determined by ELISA essentially as described above and is plotted over time in FIG. 11C.

As shown in FIG. 11A all the treatment groups exhibited comparable serum IgG depletion troughs with a maximal depletion of about 77% and comparable time to reach maximal depletion. The fastest time to reach 40% depletion was about 4 days, with the 20 mg/kg and 100 mg/kg doses maintaining lower remaining serum IgG levels through at least day 21 and day 36, respectively. As shown in FIG. 11B cx12007 administered at 20 mg/kg and 100 mg/kg doses maintained full FcRn occupancy on monocytes for 10 days and 19 days, respectively. As shown in FIG. 11C the serum concentration of cx12007 administered at 20 mg/kg and 100 mg/kg doses remains above 10 μg/mL for 10 days and 19 days, respectively. The profiles for the 100 mg/kg dose were similar for both the I.V. and S.C. routes of administration.

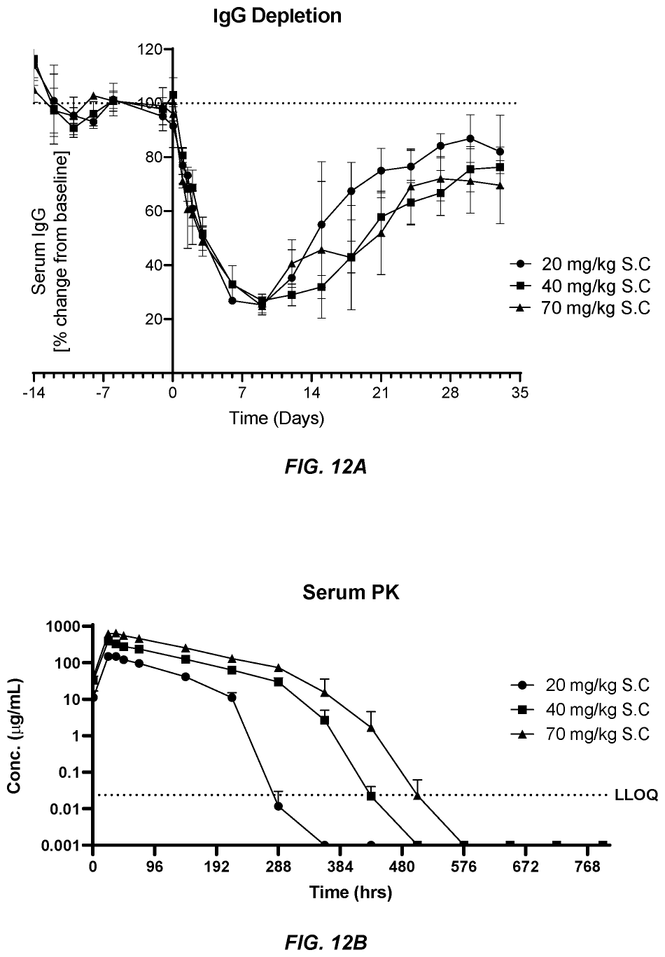

In a further study the PK and PD profiles of the humanized polypeptide cx12007 administered subcutaneously (S.C.) were evaluated at different dosages in cynomolgus monkeys. For this study naïve male cynomolgus monkeys were split into 3 treatment groups containing 3 animals per group. Groups 1, 2, and 3 were treated with a single dose of 20 mg/kg, 40 mg/kg, and 70 mg/kg of cx12007 respectively administered by S.C. at 1 mL/kg. Serum samples were collected from whole blood from all animals and all groups pre-dose, at the end of infusion, and 24, 36, 48, 72, 144, 216, 288, 360, 432, 504, 576, 648, 720, and 792 hours post-dose. Cynomolgus IgG concentration was analyzed via a NHP Isotyping Kit from Meso Scale Discovery (K15203D) as described above. The IgG concentration (as percent change in from baseline) over time for each of the treatment groups is plotted in FIG. 12A. The serum concentration of cx12007 was determined by ELISA essentially as described above and is plotted over time in FIG. 12A.

As shown in FIG. 12A all the treatment groups exhibited comparable serum IgG depletion troughs with a maximal depletion of about >70% and comparable time to reach maximal depletion. The cx12007 administered S.C. at all dose levels maintained lower remaining serum IgG levels throughout the study, with the IgG level increasing faster in the 20 mg/kg group. As shown in FIG. 12B the serum concentration of cx12007 administered S.C. at 20 mg/kg, 40 mg/kg and 70 mg/kg doses remains above 10 μg/mL for about 9 days, at least 12 days, and at least 15 days, respectively.

In these studies, administration of the humanized polypeptides targeting human FcRn and albumin was well tolerated, with no severe or serious adverse events observed. Together these data demonstrate that polypeptides targeting human FcRn and albumin are well tolerated, have a longer serum half-life, mediate faster IgG clearance and maintain lower serum IgG levels for a longer period of time than efgartigimod. These data also demonstrate that such molecules exhibit the same activity when administered subcutaneously.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

TABLE of Certain Sequences

| SEQ ID | | |
|---|---|---|
| NO | Description | Sequence |
| 1 | Human serum | MKWVTFISLL FLFSSAYSRG VFRRDAHKSE VAHRFKDLGE |
| | albumin (HSA); | ENFKALVLIA FAQYLQQCPF EDHVKLVNEV TEFAKTCVAD |
| | UniProtKB/Swiss- | ESAENCDKSL HTLFGDKLCT VATLRETYGE MADCCAKQEP |
| | Prot: P02768.2; | ERNECFLQHK DDNPNLPRLV RPEVDVMCTA FHDNEETFLK |
| | mature form: amino | KYLYEIARRH PYFYAPELLF FAKRYKAAFT ECCQAADKAA |
| | acids 25-609 | CLLPKLDELR DEGKASSAKQ RLKCASLQKF GERAFKAWAV |
| | | ARLSQRFPKA EFAEVSKLVT DLTKVHTECC HGDLLECADD |
| | | RADLAKYICE NQDSISSKLK ECCEKPLLEK SHCIAEVEND |
| | | EMPADLPSLA ADFVESKDVC KNYAEAKDVF LGMFLYEYAR |
| | | RHPDYSVVLL LRLAKTYETT LEKCCAAADP HECYAKVFDE |
| | | FKPLVEEPQN LIKQNCELFE QLGEYKFQNA LLVRYTKKVP |
| | | QVSTPTLVEV SRNLGKVGSK CCKHPEAKRM PCAEDYLSVV |
| | | LNQLCVLHEK TPVSDRVTKC CTESLVNRRP CFSALEVDET |
| | | YVPKEFNAET FTFHADICTL SEKERQIKKQ TALVELVKHK |
| | | PKATKEQLKA VMDDFAAFVE KCCKADDKET CFAEEGKKLV |
| | | AASQAALGL |
| 2 | Murine serum | MKWVTFLLLL FVSGSAFSRG VFRREAHKSE IAHRYNDLGE |
| | albumin (MSA); | QHFKGLVLIA FSQYLQKCSY DEHAKLVQEV TDFAKTCVAD |
| | UniProtKB/ Swiss- | ESAANCDKSL HTLFGDKLCA IPNLRENYGE LADCCTKQEP |
| | Prot: P07724.3; | ERNECFLQHK DDNPSLPPFE RPEAEAMCTS FKENPTTFMG |
| | mature form: amino | HYLHEVARRH PYFYAPELLY YAEQYNEILT QCCAEADKES |
| | acids 25-608 | CLTPKLDGVK EKALVSSVRQ RMKCSSMQKF GERAFKAWAV |
| | | ARLSQTFPNA DFAEITKLAT DLTKVNKECC HGDLLECADD |
| | | RAELAKYMCE NQATISSKLQ TCCDKPLLKK AHCLSEVEHD |
| | | TMPADLPAIA ADFVEDQEVC KNYAEAKDVF LGTFLYEYSR |
| | | RHPDYSVSLL LRLAKKYEAT LEKCCAEANP PACYGTVLAE |
| | | FQPLVEEPKN LVKTNCDLYE KLGEYGFQNA ILVRYTQKAP |
| | | QVSTPTLVEA ARNLGRVGTK CCTLPEDQRL PCVEDYLSAI |
| | | LNRVCLLHEK TPVSEHVTKC CSGSLVERRP CFSALTVDET |
| | | YVPKEFKAET FTFHSDICTL PEKEKQIKKQ TALAELVKHK |
| | | PKATAEQLKT VMDDFAQFLD TCCKAADKDT CFSTEGPNLV |
| | | TRCKDALA |
| 3 | Cynomolgus | MKWVTFISLL FLFSSAYSRG VFRRDTHKSE VAHRFKDLGE |
| | monkey serum | EHFKGLVLVA FSQYLQQCPF EEHVKLVNEV TEFAKTCVAD |
| | albumin (CSA); | ESAENCDKSL HTLFGDKLCT VATLRETYGE MADCCAKQEP |
| | mature form: amino | ERNECFLQHK DDNPNLPPLV RPEVDVMCTA FHDNEATFLK |
| | acids 25-608 | KYLYEVARRH PYFYAPELLF FAARYKAAFA ECCQAADKAA |
| | | CLLPKLDELR DEGKASSAKQ RLKCASLQKF GDRAFKAWAV |
| | | ARLSQKFPKA EFAEVSKLVT DLTKVHTECC HGDLLECADD |
| | | RADLAKYMCE NQDSISSKLK ECCDKPLLEK SHCLAEVEND |
| | | EMPADLPSLA ADYVESKDVC KNYAEAKDVF LGMFLYEYAR |
| | | RHPDYSVMLL LRLAKAYEAT LEKCCAAADP HECYAKVFDE |
| | | FQPLVEEPQN LVKQNCELFE QLGEYKFQNA LLVRYTKKVP |
| | | QVSTPTLVEV SRNLGKVGAK CCKLPEAKRM PCAEDYLSVV |
| | | LNRLCVLHEK TPVSEKVTKC CTESLVNRRP TALVELVKHK |
| | | YVPKAFNAET FTFHADMCTL SEKEKQVKKQ CFSALELDEA |
| | | PKATKEQLKG VMDNFAAFVE KCCKADDKEA CFAEEGPKFV |
| | | AASQAALA |
| 4 | Rat serum albumin | MKWVTFLLLL FISGSAFSRG VFRREAHKSE IAHRFKDLGE |
| | (RSA); UniProtKB/ | QHFKGLVLIA FSQYLQKCPY EEHIKLVQEV TDFAKTCVAD |
| | Swiss-Prot: | ENAENCDKSI HTLFGDKLCA IPKLRDNYGE LADCCAKQEP |
| | P02770.2; mature | ERNECFLQHK DDNPNLPPFQ RPEAEAMCTS FQENPTSFLG |
| | form: amino acids | HYLHEVARRH PYFYAPELLY YAEKYNEVLT QCCTESDKAA |
| | 25-608 | CLTPKLDAVK EKALVAAVRQ RMKCSSMQRF GERAFKAWAV |
| | | ARMSQRFPNA EFAEITKLAT DVTKINKECC HGDLLECADD |
| | | RAELAKYMCE NQATISSKLQ ACCDKPVLQK SQCLAEIEHD |
| | | NIPADLPSIA ADFVEDKEVC KNYAEAKDVF LGTFLYEYSR |
| | | RHPDYSVSLL LRLAKKYEAT LEKCCAEGDP PACYGTVLAE |
| | | FQPLVEEPKN LVKTNCELYE KLGEYGFQNA VLVRYTQKAP |
| | | QVSTPTLVEA ARNLGRVGTK CCTLPEAQRL PCVEDYLSAI |
| | | LNRLCVLHEK TPVSEKVTKC CSGSLVERRP CFSALTVDET |
| | | YVPKEFKAET FTFHSDICTL PDKEKQIKKQ TALAELVKHK |
| | | PKATEDQLKT VMGDFAQFVD KCCKAADKDN CFATEGPNLV |
| | | ARSKEALA |
| 5 | 4A01, Hz4A01v1, | GFAFSTSGMV |
| | Hz4A01v3, | |
| | Hz4A01v4, | |
| | Hz4A01v5, | |
| | Hz4A01v6, | |
| | Hz4A01v8, | |
| | Hz4A01v9, | |
| | Hz4A01v3-5, | |
| | Hz4A01v3-7, | |
| | Hz4A01v3-8 CDR1 | |
| 6 | Hz4A01v3-1, | GFTFSTSGMV |
| | Hz4A01v3-7.1k- | |
| | RITA, | |
| | Hz4A01v3-7.1k- | |
| | RITI, | |
| | Hz4A01v3-7.1k- | |
| | RITL, | |
| | Hz4A01v3-7.1k- | |
| | RITS, | |
| | Hz4A01v3-7.1k- | |
| | RITT, | |
| | Hz4A01v3-7.1k- | |
| | RITV, Hz4A01v51, | |
| | Hz4A01v51.9, | |
| | Hz4A01v51.11, | |
| | Hz4A01v51.12, | |
| | Hz4A01v51.13 | |
| | CDR1 | |
| 7 | Hz4A01v3-3 CDR1 | GFAFSTSGMA |
| 8 | Hz4A01v3-4 CDR1 | GFAFSTSGMS |
| 9 | 4A01, Hz4A01v1 | TISDDSRIIR |
| | CDR2 | |
| 10 | Hz4A01v4 CDR2 | TISDESRIIR |
| 11 | Hz4A01v5 CDR2 | TISDEGRIIR |
| 12 | Hz4A01v6 CDR2 | TISDSGRIIR |
| 13 | Hz4A01v8 CDR2 | TISDNARIIR |
| 14 | Hz4A01v9 CDR2 | TISDDTRIIR |
| 15 | Hz4A01v3 | TISDDARIIR |
| | Hz4A01v3-1, | |
| | Hz4A01v3-3, | |
| | Hz4A01v3-4, | |
| | Hz4A01v3-5, | |
| | Hz4A01v3-7, | |
| | Hz4A01v3-8, CDR2 | |
| 16 | Hz4A01v3-7.1k- | TISDDARITA |
| | RITA CDR2 | |
| 17 | Hz4A01v3-7.1k- | TISDDARITI |
| | RITI CDR2 | |
| 18 | Hz4A01v3-7.1k- | TISDDARITL |
| | RITL CDR2 | |
| 19 | Hz4A01v3-7.1k- | TISDDARITS |
| | RITS CDR2 | |
| 20 | Hz4A01v3-7.1k- | TISDDARITT |
| | RITT CDR2 | |
| 21 | Hz4A01v3-7.1k- | TISDDARITV |
| | RITV, Hz4A01v51, | |
| | Hz4A01v51.9, | |
| | Hz4A01v51.11, | |
| | Hz4A01v51.12, | |
| | Hz4A01v51.13 | |
| | CDR2 | |
| 22 | 4A01, Hz4A01v1, | YRAGSRIGSYEDYY |
| | Hz4A01v3, | |
| | Hz4A01v4, | |
| | Hz4A01v5, | |
| | Hz4A01v6, | |
| | Hz4A01v8, | |
| | Hz4A01v9, | |
| | Hz4A01v3-1, | |
| | Hz4A01v3-3, | |
| | Hz4A01v3-4, | |
| | Hz4A01v3-5, | |
| | Hz4A01v3-7, | |
| | Hz4A01v3-8, | |
| | Hz4A01v3-7.1k- | |
| | RITA, | |
| | Hz4A01v3-7.1k- | |
| | RITI, | |
| | Hz4A01v3-7.1k- | |
| | RITL, | |
| | Hz4A01v3-7.1k- | |
| | RITS, | |
| | Hz4A01v3-7.1k- | |
| | RITT, | |
| | Hz4A01v3-7.1k- | |
| | RITV, Hz4A01v51, | |
| | Hz4A01v51.9, | |
| | Hz4A01v51.11, | |
| | Hz4A01v51.12, | |
| | Hz4A01v51.13 | |
| | CDR3 | |
| 23 | 4A01 VHH | QVQLQESGGGLVQPGGSLRLSCAASGFAFSTSGMVWVRQATGKGLEWV |
| | | STISDDSRIIRYADSVNGRFTISRDNAENTLYLLMNSLKPEDTAVYYC |
| | | ATYRAGSRIGSYEDYYRGQGTQVTVSS |
| 24 | Hz4A01v1 | EVQLVESGGGEVQPGGSLRLSCAASGFAFSTSGMVWVRQAPGKGLEWV |
| | | STISDDSRIIRYAESVKGRFTISRDNAKNTLYLQMNSLRAEDTAVYYC |
| | | ATYRAGSRIGSYEDYYRGQGTLVTVKP |
| 25 | Hz4A01v3 | EVQLVESGGGEVQPGGSLRLSCAASGFAFSTSGMVWVRQAPGKGLEWV |
| | | STISDDARIIRYAESVKGRFTISRDNAKNTLYLQMNSLRAEDTAVYYC |
| | | ATYRAGSRIGSYEDYYRGQGTLVTVKP |
| 26 | Hz4A01v4 | EVQLVESGGGEVQPGGSLRLSCAASGFAFSTSGMVWVRQAPGKGLEWV |
| | | STISDESRIIRYAESVKGRFTISRDNAKNTLYLQMNSLRAEDTAVYYC |
| | | ATYRAGSRIGSYEDYYRGQGTLVTVKP |
| 27 | Hz4A01v5 | EVQLVESGGGEVQPGGSLRLSCAASGFAFSTSGMVWVRQAPGKGLEWV |
| | | STISDEGRIIRYAESVKGRFTISRDNAKNTLYLQMNSLRAEDTAVYYC |
| | | ATYRAGSRIGSYEDYYRGQGTLVTVKP |
| 28 | Hz4A01v6 | EVQLVESGGGEVQPGGSLRLSCAASGFAFSTSGMVWVRQAPGKGLEWV |
| | | STISDSGRIIRYAESVKGRFTISRDNAKNTLYLQMNSLRAEDTAVYYC |
| | | ATYRAGSRIGSYEDYYRGQGTLVTVKP |
| 29 | Hz4A01v8 | EVQLVESGGGEVQPGGSLRLSCAASGFAFSTSGMVWVRQAPGKGLEWV |
| | | STISDNARIIRYAESVKGRFTISRDNAKNTLYLQMNSLRAEDTAVYYC |
| | | ATYRAGSRIGSYEDYYRGQGTLVTVKP |
| 30 | Hz4A01v9 | EVQLVESGGGEVQPGGSLRLSCAASGFAFSTSGMVWVRQAPGKGLEWV |
| | | STISDDTRIIRYAESVKGRFTISRDNAKNTLYLQMNSLRAEDTAVYYC |
| | | ATYRAGSRIGSYEDYYRGQGTLVTVKP |
| 31 | Hz4A01v3-1 | EVQLVESGGGEVQPGGSLRLSCAASGFTFSTSGMVWVRQAPGKGLEWV |
| | | STISDDARIIRYAESVKGRFTISRDNAKNTLYLQMNSLRAEDTAVYYC |
| | | ATYRAGSRIGSYEDYYRGQGTLVTVKP |
| 32 | Hz4A01v3-3 | EVQLVESGGGEVQPGGSLRLSCAASGFAFSTSGMAWVRQAPGKGLEWV |
| | | STISDDARIIRYAESVKGRFTISRDNAKNTLYLQMNSLRAEDTAVYYC |
| | | ATYRAGSRIGSYEDYYRGQGTLVTVKP |
| 33 | Hz4A01v3-4 | EVQLVESGGGEVQPGGSLRLSCAASGFAFSTSGMSWVRQAPGKGLEWV |
| | | STISDDARIIRYAESVKGRFTISRDNAKNTLYLQMNSLRAEDTAVYYC |
| | | ATYRAGSRIGSYEDYYRGQGTLVTVKP |
| 34 | Hz4A01v3-5 | EVQLVESGGGEVQPGGSLRLSCAASGFAFSTSGMVWYRQAPGKGLEWV |
| | | STISDDARIIRYAESVKGRFTISRDNAKNTLYLQMNSLRAEDTAVYYC |
| | | ATYRAGSRIGSYEDYYRGQGTLVTVKP |
| 35 | Hz4A01v3-7 | EVQLVESGGGEVQPGGSLRLSCAASGFAFSTSGMVWVRQAPGKGLEWV |
| | | STISDDARIIRYAESVKGRFTISRDNAKNTVYLQMNSLRAEDTAVYYC |
| | | ATYRAGSRIGSYEDYYRGQGTLVTVKP |
| 36 | Hz4A01v3-8 | EVQLVESGGGEVQPGGSLRLSCAASGFAFSTSGMVWVRQAPGKGLEWV |
| | | STISDDARIIRYAESVKGRFTSRDNAKNTLYLQMNSLRAEDTAVYYC |
| | | ATYRAGSRIGSYEDYYWGQGTLVTVKP |
| 37 | Hz4A01v3-7.1k- | EVQLVESGGGEVQPGKSLRLSCAASGFTFSTSGMVWVRQAPGKGLEWV |
| | RITA | STISDDARITAYAESVKGRFTISRDNAKNTVYLQMNSLRAEDTAVYYC |
| | | ATYRAGSRIGSYEDYYRGQGTLVTVKP |
| 38 | Hz4A01v3-7.1k- | EVQLVESGGGEVQPGKSLRLSCAASGFTFSTSGMVWVRQAPGKGLEWV |
| | RITI | STISDDARITIYAESVKGRFTISRDNAKNTVYLQMNSLRAEDTAVYYC |
| | | ATYRAGSRIGSYEDYYRGQGTLVTVKP |
| 39 | Hz4A01v3-7.1k- | EVQLVESGGGEVQPGKSLRLSCAASGFTFSTSGMVWVRQAPGKGLEWV |
| | RITL | STISDDARITLYAESVKGRFTISRDNAKNTVYLQMNSLRAEDTAVYYC |
| | | ATYRAGSRIGSYEDYYRGQGTLVTVKP |
| 40 | Hz4A01v3-7.1k- | EVQLVESGGGEVQPGKSLRLSCAASGFTFSTSGMVWVRQAPGKGLEWV |
| | RITS | STISDDARITSYAESVKGRFTISRDNAKNTVYLQMNSLRAEDTAVYYC |
| | | ATYRAGSRIGSYEDYYRGQGTLVTVKP |
| 41 | Hz4A01v3-7.1k- | EVQLVESGGGEVQPGKSLRLSCAASGFTFSTSGMVWVRQAPGKGLEWV |
| | RITT | STISDDARITTYAESVKGRFTISRDNAKNTVYLQMNSLRAEDTAVYYC |
| | | ATYRAGSRIGSYEDYYRGQGTLVTVKP |
| 42 | Hz4A01v3-7.1k- | EVQLVESGGGEVQPGKSLRLSCAASGFTFSTSGMVWVRQAPGKGLEWV |
| | RITV | STISDDARITVYAESVKGRFTISRDNAKNTVYLQMNSLRAEDTAVYYC |
| | | ATYRAGSRIGSYEDYYRGQGTLVTVKP |
| 43 | Hz4A01v51 VHH | EVQLVESGGGEVQPGGSLRLSCAASGFTFSTSGMVWVRQAPGKGLEWV |
| | | STISDDARITVYAESVKGRFTISRDNAKNTVYLQMNSLRAEDTAVYYC |
| | | ATYRAGSRIGSYEDYYRGQGTLVTVKP |
| 97 | Hz4A01v51.9 VHH | QVQLLESGGGEVQPGGSLRLSCAASGFTFSTSGMVWVRQAPGKGLEWV |
| | | STISDDARITVYAESVKGRFTISRDNAKNTVYLQMNSLRAEDTAVYYC |
| | | ATYRAGSRIGSYEDYYRGQGTLVTVEP |
| 98 | Hz4A01v51.11 | QVQLLESGGGKVQPGGSLRLSCAASGFTFSTSGMVWVRQAPGKGLEWV |
| | VHH | STISDDARITVYAESVKGRFTISRDNAKNTVYLQMNSLRAEDTAVYYC |
| | | ATYRAGSRIGSYEDYYRGQGTLVTVEP |
| 99 | Hz4A01v51.12 | EVQLLESGGGKVQPGGSLRLSCAASGFTFSTSGMVWVRQAPGKGLEWV |
| | VHH | STISDDARITVYAESVKGRFTISRDNAKNTVYLQMNSLRAEDTAVYYC |
| | | ATYRAGSRIGSYEDYYRGQGTLVTVEP |
| 100 | Hz4A01v51.13 | EVQLVESGGGEVQPGGSLRLSCAASGFTFSTSGMVWVRQAPGKGLEWV |
| | VHH | STISDDARITVYAESVKGRFTISRDNAKNTVYLQMNSLRAEDTAVYYC |
| | | ATYRAGSRIGSYEDYYRGQGTLVTVRP |
| 102 | cx5009 (hz4A01v51- | EVQLVESGGGEVQPGGSLRLSCAASGFTFSTSGMVWVRQAPGKG |
| | NNT-hFc) | LEWVSTISDDARITVYAESVKGRFTISRDNAKNTVYLQMNSLRA |
| | | EDTAVYYCATYRAGSRIGSYEDYYRGQGTLVTVKPGGGGPSVEL |
| | | FPPKPKDTLMISRTPEVTCVVVDVSHEDPEVKFNWYVDGVEVHN |
| | | AKTKPREEQYNSTYRVVSVLTVLHQDWLNGKEYKCKVSNKALPA |
| | | PIEKTISKAKGQPREPQVYTLPPSRDELTKNQVNLTCLVKGFYP |
| | | SDIAVEWESNGQPENNYKTTPPVLDSDGSFFLNSTLTVDKSRWQ |
| | | QGNVFSCSVMHEALHNHYTQKSLSLSPGSGGGGSGGGGS |
| 103 | cx11956 | EVQLVESGGGEVQPGGSLRLSCAASGFTFSTSGMVWVRQAPGKG |
| | (hz4A01v51-xELL) | LEWVSTISDDARITVYAESVKGRFTISRDNAKNTVYLQMNSLRA |
| | | EDTAVYYCATYRAGSRIGSYEDYYRGQGTLVTVKPGGGGDKTHT |
| | | CPPCPAPGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALPAPIEKTISKAKGQPREPQVYTLPPSRDELTK |
| | | NQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 47 | human IgG1 Fc | DKTHTCPPC PAPELLGGPS VFLFPPKPKD TLMISRTPEV |
| | region | TCVVVDVSHE DPEVKENWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 48 | human IgG1 Fc | DKTHTCPPCP APGGPSVFLF PPKPKDTLMI SRTPEVTCVV |
| | xELL | VDVSHEDPEV KFNWYVDGVE VHNAKTKPRE EQYNSTYRVV |
| | | SVLTVLHQDW LNGKEYKCKV SNKALPAPIE KTISKAKGQP |
| | | REPQVYTLPP SRDELTKNQV SLTCLVKGFY PSDIAVEWES |
| | | NGQPENNYKT TPPVLDSDGS FFLYSKLTVD KSRWQQGNVF |
| | | SCSVMHEALH NHYTQKSLSL SPGK |
| 49 | Fc region M252Y | DKTHTCPPCP APELLGGPSV FLFPPKPKDT LYISRTPEVT |
| | and M428V (YV) | CVVVDVSHED PEVKFNWYVD GVEVHNAKTK PREEQYNSTY |
| | S354C T366W knob | RVVSVLTVLH QDWLNGKEYK CKVSNKALPA PIEKTISKAK |
| | | GQPREPQVYT LPPCRDELTK NQVSLWCLVK GFYPSDIAVE |
| | | WESNGQPENN YKTTPPVLDS DGSFFLYSKL TVDKSRWQQG |
| | | NVFSCSVVHE ALHNHYTQKS LSLSPGK |
| 50 | Fc region M252Y, | DKTHTCPPCP APELLGGPSV FLFPPKPKDT LYISRTPEVT |
| | M428V, H435R | CVVVDVSHED PEVKFNWYVD GVEVHNAKTK PREEQYNSTY |
| | (YVR) T366S, | RVVSVLTVLH QDWLNGKEYK CKVSNKALPA PIEKTISKAK |
| | L368A, Y407V hole | GQPREPQVCT LPPSRDELTK NQVSLSCAVK GFYPSDIAVE |
| | | WESNGQPENN YKTTPPVLDS DGSFFLVSKL TVDKSRWQQG |
| | | NVFSCSVVHE ALHNRYTQKS LSLSPGK |
| 51 | Fc region xELL | DKTHTC PPCPAPGGPS VFLFPPKPKD TLMISRTPEV |
| | H435R | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNRYTQK SLSLSPGK |
| 52 | Fc region xELL | DKTHTC PPCPAPGGPS VFLFPPKPKD TLYISRTPEV |
| | M252Y and M428V | TCVVVDVSHE DPEVKFNWYV DGVEVHNAKT KPREEQYNST |
| | (YV) | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVVH EALHNHYTQK SLSLSPGK |
| 53 | Fc region xELL | DKTHTC PPCPAPGGPS VFLFPPKPKD TLYISRTPEV |
| | M252Y and M428L | TCVVVDVSHE DPEVKFNWYV DGVEVHNAKT KPREEQYNST |
| | (YL) | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVLH EALHNHYTQK SLSLSPGK |
| 54 | Fc region xELL | DKTHTC PPCPAPGGPS VFLFPPKPKD TLYISRTPEV |
| | M252Y, M428L, | TCVVVDVSHE DPEVKFNWYV DGVEVHNAKT KPREEQYNST |
| | H435R (YLR) | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVLH EALHNRYTQK SLSLSPGK |
| 55 | Fc region xELL | DKTHTC PPCPAPGGPS VFLFPPKPKD TLYISRTPEV |
| | M252Y, M428V, | TCVVVDVSHE DPEVKFNWYV DGVEVHNAKT KPREEQYNST |
| | H435R (YVR) | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVVH EALHNRYTQK SLSLSPGK |
| 56 | Fc region xELL | DKTHTC PPCPAPGGPS VFLFPPKPKD TLMISRTPEV |
| | S354C T366W knob | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPCRDELT KNQVSLWCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 57 | Fc region xELL | DKTHTC PPCPAPGGPS VFLFPPKPKD TLMISRTPEV |
| | H435R S354C | TCVVVDVSHE DPEVKFNWYV DGVEVHNAKT KPREEQYNST |
| | T366W knob | YRVVSVLTVL HQDWLNGKEY KCKVSNKALP APIEKTISKA |
| | | KGQPREPQVY TLPPCRDELT KNQVSLWCLV KGFYPSDIAV |
| | | EWESNGQPEN NYKTTPPVLD SDGSFFLYSK LTVDKSRWQQ |
| | | GNVFSCSVMH EALHNRYTQK SLSLSPGK |
| 58 | Fc region xELL | DKTHTCPPCPAPGGPSVFLFPPKPKDTLYISRTPEVTCVVVDVSHEDP |
| | M252Y and M428V | EVKFNWYVDGVEVHNAKTKPREEQYNSTYRVVSVLTVLHQDWLNGKEY |
| | (YV) S354C T366W | KCKVSNKALPAPIEKTISKAKGQPREPQVYTLPPCRDELTKNQVSLWC |
| | knob | LVKGFYPSDIAVEWESNGQPENNYKTTPPVLDSDGSFFLYSKLTVDKS |
| | | RWQQGNVFSCSVVHEALHNHYTQKSLSLSPGK |
| 59 | Fc region xELL | DKTHTCPPCPAPGGPSVFLFPPKPKDTLYISRTPEVTCVVVDVSHEDP |
| | M252Y and M428L | EVKFNWYVDGVEVHNAKTKPREEQYNSTYRVVSVLTVLHQDWLNGKEY |
| | (YL) S354C T366W | KCKVSNKALPAPIEKTISKAKGQPREPQVYTLPPCRDELTKNQVSLWC |
| | knob | LVKGFYPSDIAVEWESNGQPENNYKTTPPVLDSDGSFFLYSKLTVDKS |
| | | RWQQGNVFSCSVLHEALHNHYTQKSLSLSPGK |
| 60 | Fc region xELL | DKTHTCPPCPAPGGPSVFLFPPKPKDTLYISRTPEVTCVVVDVSHEDP |
| | M252Y, M428L, | EVKFNWYVDGVEVHNAKTKPREEQYNSTYRVVSVLTVLHQDWLNGKEY |
| | H435R (YLR) | KCKVSNKALPAPIEKTISKAKGQPREPQVYTLPPCRDELTKNQVSLWC |
| | S354C T366W knob | LVKGFYPSDIAVEWESNGQPENNYKTTPPVLDSDGSFFLYSKLTVDKS |
| | | RWQQGNVFSCSVLHEALHNRYTQKSLSLSPGK |
| 61 | Fc region xELL | DKTHTCPPCPAPGGPSVFLFPPKPKDTLYISRTPEVTCVVVDVSHEDP |
| | M252Y, M428V, | EVKFNWYVDGVEVHNAKTKPREEQYNSTYRVVSVLTVLHQDWLNGKEY |
| | H435R (YVR) | KCKVSNKALPAPIEKTISKAKGQPREPQVYTLPPCRDELTKNQVSLWC |
| | S354C T366W knob | LVKGFYPSDIAVEWESNGQPENNYKTTPPVLDSDGSFFLYSKLTVDKS |
| | | RWQQGNVFSCSVVHEALHNRYTQKSLSLSPGK |
| 62 | Fc region xELL | DKTHTCPPCPAPGGPSVFLFPPKPKDTLMISRTPEVTCVVVDVSHEDP |
| | T366S, L368A, | EVKFNWYVDGVEVHNAKTKPREEQYNSTYRVVSVLTVLHQDWLNGKEY |
| | Y407V hole | KCKVSNKALPAPIEKTISKAKGQPREPQVCTLPPSRDELTKNQVSLSC |
| | | AVKGFYPSDIAVEWESNGQPENNYKTTPPVLDSDGSFFLVSKLTVDKS |
| | | RWQQGNVFSCSVMHEALHNHYTQKSLSLSPGK |
| 63 | Fc region xELL | DKTHTCPPCPAPGGPSVFLFPPKPKDTLMISRTPEVTCVVVDVSHEDP |
| | H435R, T366S, | EVKFNWYVDGVEVHNAKTKPREEQYNSTYRVVSVLTVLHQDWLNGKEY |
| | L368A, Y407V hole | KCKVSNKALPAPIEKTISKAKGQPREPQVCTLPPSRDELTKNQVSLSC |
| | | AVKGFYPSDIAVEWESNGQPENNYKTTPPVLDSDGSFFLVSKLTVDKS |
| | | RWQQGNVFSCSVMHEALHNRYTQKSLSLSPGK |
| 64 | Fc region xELL | DKTHTCPPCPAPGGPSVFLFPPKPKDTLYISRTPEVTCVVVDVSHEDP |
| | M252Y and M428V | EVKFNWYVDGVEVHNAKTKPREEQYNSTYRVVSVLTVLHQDWLNGKEY |
| | (YV) T366S, | KCKVSNKALPAPIEKTISKAKGQPREPQVCTLPPSRDELTKNQVSLSC |
| | L368A, Y407V hole | AVKGFYPSDIAVEWESNGQPENNYKTTPPVLDSDGSFFLVSKLTVDKS |
| | | RWQQGNVFSCSVVHEALHNHYTQKSLSLSPGK |
| 65 | Fc region xELL | DKTHTCPPCPAPGGPSVFLFPPKPKDTLYISRTPEVTCVVVDVSHEDP |
| | M252Y and M428L | EVKFNWYVDGVEVHNAKTKPREEQYNSTYRVVSVLTVLHQDWLNGKEY |
| | (YL) T366S, | KCKVSNKALPAPIEKTISKAKGQPREPQVCTLPPSRDELTKNQVSLSC |
| | L368A, Y407V hole | AVKGFYPSDIAVEWESNGQPENNYKTTPPVLDSDGSFFLVSKLTVDKS |
| | | RWQQGNVFSCSVLHEALHNHYTQKSLSLSPGK |
| 66 | Fc region xELL | DKTHTCPPCPAPGGPSVFLFPPKPKDTLYISRTPEVTCVVVDVSHEDP |
| | M252Y, M428L, | EVKFNWYVDGVEVHNAKTKPREEQYNSTYRVVSVLTVLHQDWLNGKEY |
| | H435R (YLR) | KCKVSNKALPAPIEKTISKAKGQPREPQVCTLPPSRDELTKNQVSLSC |
| | T366S, L368A, | AVKGFYPSDIAVEWESNGQPENNYKTTPPVLDSDGSFFLVSKLTVDKS |
| | Y407V hole | RWQQGNVFSCSVLHEALHNRYTQKSLSLSPGK |
| 67 | Fc region xELL | DKTHTCPPCPAPGGPSVFLFPPKPKDTLYISRTPEVTCVVVDVSHEDP |
| | M252Y, M428V, | EVKFNWYVDGVEVHNAKTKPREEQYNSTYRVVSVLTVLHQDWLNGKEY |
| | H435R (YVR) | KCKVSNKALPAPIEKTISKAKGQPREPQVCTLPPSRDELTKNQVSLSC |
| | T366S, L368A, | AVKGFYPSDIAVEWESNGQPENNYKTTPPVLDSDGSFFLVSKLTVDKS |
| | Y407V hole | RWQQGNVFSCSVVHEALHNRYTQKSLSLSPGK |
| 68 | Fc region S364N, | GGPSVFLFPPKPKDTLMISRTPEVTCVVVDVSHEDPEVKFNWYVDGVE |
| | Y407N, and K409T | VHNAKTKPREEQYNSTYRVVSVLTVLHQDWLNGKEYKCKVSNKALPAP |
| | (NNT) | IEKTISKAKGQPREPQVYTLPPSRDELTKNQVNLTCLVKGFYPSDIAV |
| | | EWESNGQPENNYKTTPPVLDSDGSFFLNSTLTVDKSRWQQGNVFSCSV |
| | | MHEALHNHYTQKSLSLSPG |
| 69 | cx11383 | EVQLLESGGGEVQPGGSLRLSCTASGSIANVDMMAWHRQAPGKQ |
| | (B10a.v1 VHH x | REPIAVINDGGSTSYADSVKGRFTISRDNAKNTLYLQMSSLRAE |
| | hz4A01v51 VHH) | DTAVYYCNTATWRGGERYADWGQGTLVTVKPGSGGGSEVQLVES |
| | | GGGEVQPGGSLRLSCAASGFTFSTSGMVWVRQAPGKGLEWVSTI |
| | | SDDARITVYAESVKGRFTISRDNAKNTVYLQMNSLRAEDTAVYY |
| | | CATYRAGSRIGSYEDYYRGQGTLVTVKPGG |
| 70 | cx11558 | EVQLLESGGGEVQPGGSLRLSCAASGSIANVDLMAWHRQAPGKQ |
| | (B10a.v2.1 VHH x | REPIAVINDAGSTYYAESVKGRFTISRDNAKNTLYLQMNSLRAE |
| | hz4A01v51 VHH) | DTAVYYCNTATWRGGERYADWGQGTLVTVKPGSGGGSEVQLVES |
| | | GGGEVQPGGSLRLSCAASGFTFSTSGMVWVRQAPGKGLEWVSTI |
| | | SDDARITVYAESVKGRFTISRDNAKNTVYLQMNSLRAEDTAVYY |
| | | CATYRAGSRIGSYEDYYRGQGTLVTVKPGG |
| 71 | cx12003 | QVQLLESGGGEVQPGGSLRLSCAASGSIANVDLMAWHRQAPGKQ |
| | (B10a.v2.2 VHH x | REPIAVINDAGSTYYAESVKGRFTISRDNAKNTLYLQMNSLRAE |
| | hz4A01v51.9 VHH) | DTAVYYCNTATWRGGERYADWGQGTLVTVEPGSGGGSQVQLLES |
| | | GGGEVQPGGSLRLSCAASGFTFSTSGMVWVRQAPGKGLEWVSTI |
| | | SDDARITVYAESVKGRFTISRDNAKNTVYLQMNSLRAEDTAVYY |
| | | CATYRAGSRIGSYEDYYRGQGTLVTVEPGG |
| 72 | cx12006 | QVQLLESGGGKVQPGGSLRLSCAASGSIANVDLMAWHRQAPGKQ |
| | (B10a.v2.3 VHH x | REPIAVINDAGSTYYAESVKGRFTISRDNAKNTLYLQMNSLRAE |
| | hz4A01v51.11 VHH | DTAVYYCNTATWRGGERYADWGQGTLVTVEPGSGGGSQVQLLES |
| | | GGGKVQPGGSLRLSCAASGFTFSTSGMVWVRQAPGKGLEWVSTI |
| | | SDDARITVYAESVKGRETISRDNAKNTVYLQMNSLRAEDTAVYY |
| | | CATYRAGSRIGSYEDYYRGQGTLVTVEPGG |
| 73 | cx12007 | EVQLLESGGGKVQPGGSLRLSCAASGSIANVDLMAWHRQAPGKQ |
| | (B10a.v2.4 VHH x | REPIAVINDAGSTYYAESVKGRFTISRDNAKNTLYLQMNSLRAE |
| | hz4A01v51.12 VHH | DTAVYYCNTATWRGGERYADWGQGTLVTVEPGSGGGSEVQLLES |
| | | GGGKVQPGGSLRLSCAASGFTFSTSGMVWVRQAPGKGLEWVSTI |
| | | SDDARITVYAESVKGRFTISRDNAKNTVYLQMNSLRAEDTAVYY |
| | | CATYRAGSRIGSYEDYYRGQGTLVTVEPGG |
| 74 | cx12008 | EVQLLESGGGEVQPGGSLRLSCAASGSIANVDLMAWHRQAPGKQ |
| | (B10a.v2.5 VHH x | REPIAVINDAGSTYYAESVKGRFTISRDNAKNTLYLQMNSLRAE |
| | hz4A01v51.13 | DTAVYYCNTATWRGGERYADWGQGTLVTVRPGSGGGSEVQLVES |
| | VHH) | GGGEVQPGGSLRLSCAASGFTFSTSGMVWVRQAPGKGLEWVSTI |
| | | SDDARITVYAESVKGRETISRDNAKNTVYLQMNSLRAEDTAVYY |
| | | CATYRAGSRIGSYEDYYRGQGTLVTVRPGG |
| 75 | cx11385 | EVQLLESGGGEVQPGGSLRLSCTASGSIANVDMMAWHRQAPGKQ |
| | (B10a.v1 VHH x | REPIAVINDGGSTSYADSVKGRFTISRDNAKNTLYLQMSSLRAE |
| | B10a.v1 VHH x | DTAVYYCNTATWRGGERYADWGQGTLVTVKPGSGGGSEVQLLES |
| | hz4A01v51 VHH) | GGGEVQPGGSLRLSCTASGSIANVDMMAWHRQAPGKQREPIAVI |
| | | NDGGSTSYADSVKGRFTISRDNAKNTLYLQMSSLRAEDTAVYYC |
| | | NTATWRGGERYADWGQGTLVTVKPGSGGGSEVQLVESGGGEVQP |
| | | GGSLRLSCAASGFTFSTSGMVWVRQAPGKGLEWVSTISDDARIT |
| | | VYAESVKGRFTISRDNAKNTVYLQMNSLRAEDTAVYYCATYRAG |
| | | SRIGSYEDYYRGQGTLVTVKPGG |
| 76 | cx11642 (B10a.v2.1 | EVQLLESGGGEVQPGGSLRLSCAASGSIANVDLMAWHRQAPGKQ |
| | VHH x B10a.v2.1 | REPIAVINDAGSTYYAESVKGRFTISRDNAKNTLYLQMNSLRAE |
| | VHH x hz4A01v51 | DTAVYYCNTATWRGGERYADWGQGTLVTVKPGSGGGSEVQLLES |
| | VHH) | GGGEVQPGGSLRLSCAASGSIANVDLMAWHRQAPGKQREPIAVI |
| | | NDAGSTYYAESVKGRFTISRDNAKNTLYLQMNSLRAEDTAVYYC |
| | | NTATWRGGERYADWGQGTLVTVKPGSGGGSEVQLVESGGGEVQP |
| | | GGSLRLSCAASGFTFSTSGMVWVRQAPGKGLEWVSTISDDARIT |
| | | VYAESVKGRFTISRDNAKNTVYLQMNSLRAEDTAVYYCATYRAG |
| | | SRIGSYEDYYRGQGTLVTVKPGG |
| 77 | cx12032 (B10a.v2.1 | EVQLLESGGGEVQPGGSLRLSCAASGSIANVDLMAWHRQAPGKQ |
| | VHH x hz4A01v51 | REPIAVINDAGSTYYAESVKGRFTISRDNAKNTLYLQMNSLRAE |
| | VHH) | DTAVYYCNTATWRGGERYADWGQGTLVTVKPEVQLVESGGGEVQ |
| | | PGGSLRLSCAASGFTFSTSGMVWVRQAPGKGLEWVSTISDDARI |
| | | TVYAESVKGRFTISRDNAKNTVYLQMNSLRAEDTAVYYCATYRA |
| | | GSRIGSYEDYYRGQGTLVTVKPGG |
| 78 | FcRn ECD | AESHLSLLYHLTAVSSPAPGTPAFWVSGWLGPQQYLSYNSLRGE |
| | | AEPCGAWVWENQVSWYWEKETTDLRIKEKLFLEAFKALGGKGPY |
| | | TLQGLLGCELGPDNTSVPTAKFALNGEEFMNFDLKQGTWGGDWP |
| | | EALAISQRWQQQDKAANKELTFLLFSCPHRLREHLERGRGNLEW |
| | | KEPPSMRLKARPSSPGFSVLTCSAFSFYPPELQLRELRNGLAAG |
| | | TGQGDFGPNSDGSFHASSSLTVKSGDEHHYCCIVQHAGLAQPLR |
| | | VELESPAKSS |
| 79 | Beta 2 microglobulin | MSRSVALAVLALLSLSGLEAIQRTPKIQVYSRHPAENGKSNELN |
| | | CYVSGFHPSDIEVDLLKNGERIEKVEHSDLSFSKDWSFYLLYYT |
| | | EFTPTEKDEYACRVNHVTLSQPKIVKWDRDM |
| 80 | FcRn B10a.v1 CDR1 | GSIANVDMMA |
| 81 | FcRn B10a.v2 CDR1 | GSIANVDLMA |
| 83 | FcRn B10a.v1 CDR2 | VINDGGSTS |
| 84 | FcRn B10a.v2 CDR2 | VINDAGSTY |
| 85 | FcRn B10a.v1, | ATWRGGERYAD |
| | B10a.v2 CDR3 | |
| 86 | FcRn B10a.v1 VHH | EVQLLESGGGEVQPGGSLRLSCTASGSIANVDMMAWHRQAPGKQ |
| | | REPIAVINDGGSTSYADSVKGRFTISRDNAKNTLYLQMSSLRAE |
| | | DTAVYYCNTATWRGGERYADWGQGTLVTVKP |
| 87 | FcRn B10a.v2.1 VHH | EVQLLESGGGEVQPGGSLRLSCAASGSIANVDLMAWHRQAPGKQ |
| | | REPIAVINDAGSTYYAESVKGRFTISRDNAKNTLYLQMNSLRAE |
| | | DTAVYYCNTATWRGGERYADWGQGTLVTVKP |
| 88 | FcRn B10a.v2.2 VHH | QVQLLESGGGEVQPGGSLRLSCAASGSIANVDLMAWHRQAPGKQ |
| | | REPIAVINDAGSTYYAESVKGRFTISRDNAKNTLYLQMNSLRAE |
| | | DTAVYYCNTATWRGGERYADWGQGTLVTVEP |
| 89 | FcRn B10a.v2.3 VHH | QVQLLESGGGKVQPGGSLRLSCAASGSIANVDLMAWHRQAPGKQ |
| | | REPIAVINDAGSTYYAESVKGRFTISRDNAKNTLYLQMNSLRAE |
| | | DTAVYYCNTATWRGGERYADWGQGTLVTVEP |
| 90 | FcRn B10a.v2.4 VHH | EVQLLESGGGKVQPGGSLRLSCAASGSIANVDLMAWHRQAPGKQ |
| | | REPIAVINDAGSTYYAESVKGRFTISRDNAKNTLYLQMNSLRAE |
| | | DTAVYYCNTATWRGGERYADWGQGTLVTVEP |
| 91 | FcRn B10a.v2.5 VHH | EVQLLESGGGEVQPGGSLRLSCAASGSIANVDLMAWHRQAPGKQ |
| | | REPIAVINDAGSTYYAESVKGRFTISRDNAKNTLYLQMNSLRAE |
| | | DTAVYYCNTATWRGGERYADWGQGTLVTVRP |
| 92 | UniProtKB-P55899 | MGVPRPQPWA LGLLLELLPG SLGAESHLSL LYHLTAVSSP |
| | (FCGRN_HUMAN) | APGTPAFWVS GWLGPQQYLS YNSLRGEAEP CGAWVWENQV |
| | (human) | SWYWEKETTD LRIKEKLFLE AFKALGGKGP YTLQGLLGCE |
| | | LGPDNTSVPT AKFALNGEEF MNEDLKQGTW GGDWPEALAI |
| | | SQRWQQQDKA ANKELTELLF SCPHRLREHL ERGRGNLEWK |
| | | EPPSMRLKAR PSSPGFSVLT CSAFSFYPPE LQLRELRNGL |
| | | AAGTGQGDFG PNSDGSFHAS SSLTVKSGDE HHYCCIVQHA |
| | | GLAQPLRVEL ESPAKSSVLV VGIVIGVLLL TAAAVGGALL |
| | | WRRMRSGLPA PWISLRGDDT GVLLPTPGEA QDADLKDVNV |
| | | IPATA |
| 93 | UniProtKB-Q61559 | MGMPLPWALS LLLVLLPQTW GSETRPPLMY HLTAVSNPST |
| | (FCGRN_MOUSE) | GLPSFWATGW LGPQQYLTYN SLRQEADPCG AWMWENQVSW |
| | (mouse) | YWEKETTDLK SKEQLFLEAL KTLEKILNGT YTLQGLIGCE |
| | | LASDNSSVPT AVFALNGEEF MKENPRIGNW TGEWPETEIV |
| | | ANLWMKQPDA ARKESEFLLN SCPERLLGHL ERGRRNLEWK |
| | | EPPSMRLKAR PGNSGSSVLT CAAFSFYPPE LKFRELRNGL |
| | | ASGSGNCSTG PNGDGSFHAW SLLEVKRGDE HHYQCQVEHE |
| | | GLAQPLTVDL DSSARSSVPV VGIVLGLLLV VVAIAGGVLL |
| | | WGRMRSGLPA PWLSLSGDDS GDLLPGGNLP PEAEPQGANA |
| | | FPATS |
| 94 | UniProtKB-Q8SPV9 | MRVPRPQPWA LGLLLFLLPG SLGAESHLSL LYHLTAVSSP |
| | (FCGRN_MACFA) | APGTPAFWVS GWLGPQQYLS YDSLRGQAEP CGAWVWENQV |
| | (Cynomolgus | SWYWEKETTD LRIKEKLFLE AFKALGGKGP YTLQGLLGCE |
| | monkey) | LSPDNTSVPT AKFALNGEEF MNEDLKQGTW GGDWPEALAI |
| | | SQRWQQQDKA ANKELTELLF SCPHRLREHL ERGRGNLEWK |
| | | EPPSMRLKAR PGNPGFSVLT CSAFSFYPPE LQLRFLRNGM |
| | | AAGTGQGDFG PNSDGSFHAS SSLTVKSGDE HHYCCIVQHA |
| | | GLAQPLRVEL ETPAKSSVLV VGIVIGVLLL TAAAVGGALL |
| | | WRRMRSGLPA PWISLRGDDT GSLLPTPGEA QDADSKDINV |
| | | IPATA |
| 95 | UniProtKB- | MGMSQPGVLL SLLLVLLPQT WGAEPRLPLM YHLAAVSDLS |
| | A0A816AFJ8 | WLGAQQYLTY NNLRQEADPC GAWIWENQVS WYWEKETTDL |
| | (A0A816AFJ8_RAT) | KSKEQLFLEA IRTLENQING TFTLQGLLGC ELAPDNSSLP |
| | (rat) | TAVFALNGEE FMRFNPRTGN WSGEWPETDI VGNLWMKQPE |
| | | AARKESEFLL TSCPERLLGH LERGRQNLEW KEPPSMRLKA |
| | | RPGNSGSSVL TCAAFSFYPT GPNGDGSFHA WSLLEVKRGD |
| | | EHHYQCQVEH EGLAQPLTVD LDSPARSSVP VVGIILGLLL |
| | | VVVAIAGGVL LWNRMRSGLP APWLSLSGDD SGDLLPGGNL |
| | | PPEAEPQGVN AFPATS |
| 96 | UniProtKB-F5H610 | MSRSVALAVL ALLSLSGLEA IQRTPKIQVY SRHPADIEVD |
| | (F5H610_HUMAN) | LLKNGERIEK VEHSDLSFSK DWSFYLLYYT EFTPTEKDEY |
| | Human B2m | ACRVNHVTLS QPKIVKWDRD M |
| 101 | Linker | GSGGGS |
| 104 | VH3-23*04 | EVQLVESGGGLVQPGGSLRLSCAASGFTFSSYAMSWVRQAPGKG |
| | | LEWVSAISGSGGSTYYADSVKGRFTISRDNSKNTLYLQMNSLRA |
| | | EDTAVYYC |

What is claimed is:

1. A polypeptide comprising at least one VHH domain that binds FcRn, wherein at least one VHH domain that binds FcRn comprises a CDR1 sequence selected from SEQ ID NOs: 80-81, a CDR2 sequence selected from SEQ ID NOs: 83-84, and a CDR3 sequence of SEQ ID NO: 85.

2. The polypeptide of claim 1, wherein each VHH domain that binds FcRn comprises, independently, a CDR1 sequence selected from SEQ ID NOs: 80-81, a CDR2 sequence selected from SEQ ID NOs: 83-84, and a CDR3 sequence of SEQ ID NO: 85.

3. The polypeptide of claim 1 or claim 2, wherein at least one VHH domain that binds FcRn comprises CDR1, CDR2, and CDR3 sequences selected from: SEQ ID NOs: 80, 83, and 85 SEQ ID NOs: 81, 83, and 85; and SEQ ID NOs: 81, 84, and 85.

4. The polypeptide of claim 3, wherein each VHH domain that binds FcRn comprises, independently, CDR1, CDR2, and CDR3 sequences selected from: SEQ ID NOs: 80, 83, and 85 SEQ ID NOs: 81, 83, and 85; and SEQ ID NOs: 81, 84, and 85.

5. The polypeptide of any one of claims 1-4, wherein at least one VHH domain that binds FcRn is humanized.

6. The polypeptide of claim 5, wherein each VHH domain that binds FcRn is humanized.

7. The polypeptide of any one of claims 1-6, wherein at least one VHH domain that binds FcRn comprises a sequence at least 85%, at least 90%, at least 95%, at least 96%, at least 97%, at least 98%, at least 99% identical to a sequence selected from SEQ ID NOs: 86-93.

8. The polypeptide of claim 7, wherein each VHH domain that binds FcRn comprises a sequence at least 85%, at least 90%, at least 95%, at least 96%, at least 97%, at least 98%, at least 99% identical to a sequence selected from SEQ ID NOs: 86-93.

9. The polypeptide of any one of claims 1-7, wherein at least one VHH domain that binds FcRn comprises a sequence selected from SEQ ID NOs: 86-93.

10. The polypeptide of any one of claims 1-9, wherein each VHH domain that binds FcRn comprises a sequence selected from SEQ ID NOs: 86-93.

11. The polypeptide of any one of claims 1-10, wherein at least one VHH domain that binds FcRn binds human FcRn with an affinity of less than 5 nM, less than 2 nM, less than 1 nM, or less than 0.5 nM.

12. The polypeptide of any one of claims 1-11, wherein each VHH domain that binds FcRn binds human FcRn with an affinity of less than 5 nM, less than 2 nM, less than 1 nM, or less than 0.5 nM.

13. The polypeptide of any one of claims 1-12, wherein at least one VHH domain that binds FcRn binds human FcRn at pH 6 and at pH 7.4.

14. The polypeptide of any one of claims 1-13, wherein each VHH domain that binds FcRn binds human FcRn at pH 6 and at pH 7.4.

15. The polypeptide of any one of claims 1-14, wherein at least one VHH domain that binds FcRn blocks binding of human IgG to human FcRn.

16. The polypeptide of any one of claims 1-15, wherein each VHH domain that binds FcRn blocks binding of human IgG to human FcRn.

17. The polypeptide of any one of claims 1-16, wherein the polypeptide comprises at least one VHH domain that binds albumin.

18. The polypeptide of claim 17, wherein at least one VHH domain that binds albumin comprises a CDR1 sequence selected from SEQ ID NOs: 5-8, a CDR2 sequence selected from SEQ ID NOs: 9-21, and a CDR3 sequence of SEQ ID NO: 22.

19. That polypeptide of claim 17, wherein each VHH domain that binds albumin comprises, independently, a CDR1 sequence selected from SEQ ID NOs: 5-8, a CDR2 sequence selected from SEQ ID NOs: 9-21, and a CDR3 sequence of SEQ ID NO: 22.

20. The polypeptide of claim 17 or claim 18, wherein at least one VHH domain that binds albumin comprises CDR1, CDR2, and CDR3 sequences selected from: SEQ ID NOs: 5, 9, and 22; SEQ ID NOs: 5, 10, and 22; SEQ ID NOs: 5, 11, and 22; SEQ ID NOs: 5, 12, and 22; SEQ ID NOs: 5, 13, and 22; SEQ ID NOs: 5, 14, and 22; SEQ ID NOs: 5, 15, and 22; SEQ ID NOs: 6, 15, and 22; SEQ ID NOs: 7, 15, and 22; SEQ ID NOs: 8, 15, and 22; SEQ ID NOs: 6, 16, and 22; SEQ ID NOs: 6, 17, and 22; SEQ ID NOs: 6, 18, and 22; SEQ ID NOs: 6, 19, and 22; SEQ ID NOs: 6, 20, and 22; and SEQ ID NOs: 6, 21, and 22.

21. The polypeptide of claim 17 or claim 18, wherein each VHH domain that binds albumin comprises, independently, CDR1, CDR2, and CDR3 sequences selected from: SEQ ID NOs: 5, 9, and 22; SEQ ID NOs: 5, 10, and 22; SEQ ID NOs: 5, 11, and 22; SEQ ID NOs: 5, 12, and 22; SEQ ID NOs: 5, 13, and 22; SEQ ID NOs: 5, 14, and 22; SEQ ID NOs: 5, 15, and 22; SEQ ID NOs: 6, 15, and 22; SEQ ID NOs: 7, 15, and 22; SEQ ID NOs: 8, 15, and 22; SEQ ID NOs: 6, 16, and 22; SEQ ID NOs: 6, 17, and 22; SEQ ID NOs: 6, 18, and 22; SEQ ID NOs: 6, 19, and 22; SEQ ID NOs: 6, 20, and 22; and SEQ ID NOs: 6, 21, and 22.

22. The polypeptide of any one of claims 17-21, wherein at least one VHH domain that binds albumin is humanized.

23. The polypeptide of claim 22, wherein each VHH domain that binds albumin is humanized.

24. The polypeptide of any one of claims 17-23, wherein at least one VHH domain that binds albumin comprises a sequence at least 85%, at least 90%, at least 95%, at least 96%, at least 97%, at least 98%, at least 99% identical to a sequence selected from SEQ ID NOs: 23-43 and 97-100.

25. The polypeptide of any one of claims 17-23, wherein each VHH domain that binds albumin comprises a sequence at least 85%, at least 90%, at least 95%, at least 96%, at least 97%, at least 98%, at least 99% identical to a sequence selected from SEQ ID NOs: 23-43 and 97-100.

26. The polypeptide of any one of claims 17-23, wherein at least one VHH domain that binds albumin comprises a sequence selected from SEQ ID NOs: 23-43 and 97-100.

27. The polypeptide of any one of claims 17-23, wherein each VHH domain that binds albumin comprises a sequence selected from SEQ ID NOs: 23-43 and 97-100.

28. The polypeptide of any one of claims 17-27, wherein the polypeptide comprises a sequence at least 85%, at least 90%, at least 95%, at least 96%, at least 97%, at least 98%, or at least 99% identical to a sequence selected from SEQ ID NOs: 69-77.

29. The polypeptide of any one of claims 17-27, wherein the polypeptide comprises a sequence selected from SEQ ID NOs: 69-77.

30. The polypeptide of any one of claims 17-29, wherein at least one VHH domain that binds albumin binds human albumin and at least one albumin selected from cynomolgus monkey, mouse, and rat albumin.

31. The polypeptide of any one of claims 17-29, wherein each VHH domain that binds albumin binds human albumin and at least one albumin selected from cynomolgus monkey, mouse, and rat albumin.

32. The polypeptide of any one of claims 17-29, wherein at least one VHH domain that binds albumin binds human, cynomolgus monkey, mouse, and rat albumin.

33. The polypeptide of any one of claims 17-29, wherein each VHH domain that binds albumin binds human, cynomolgus monkey, mouse, and rat albumin.

34. The polypeptide of any one of claims 17-33, wherein at least one VHH domain that binds albumin binds human albumin with an affinity of less than 5 nM, less than 2 nM, less than 1 nM, or less than 0.5 nM.

35. The polypeptide of any one of claims 17-34, wherein at least one VHH domain that binds albumin binds each of human, cynomolgus monkey, mouse, and rat albumin with an affinity of less than 5 nM, less than 2 nM, less than 1 nM, or less than 0.5 nM.

36. The polypeptide of any one of claims 17-35, wherein each VHH domain that binds albumin binds human albumin with an affinity of less than 5 nM, less than 2 nM, less than 1 nM, or less than 0.5 nM.

37. The polypeptide of any one of claims 17-63, wherein each VHH domain that binds albumin binds each of human, cynomolgus monkey, mouse, and rat albumin with an affinity of less than 5 nM, less than 2 nM, less than 1 nM, or less than 0.5 nM.

38. The polypeptide of any one of claims 17-37, wherein the each VHH domain that binds albumin does not bind albumin domain 3.

39. The polypeptide of any one of claims 17-38, wherein the each VHH domain that binds albumin does not interfere with binding of albumin to FcRn.

40. The polypeptide of any one of the preceding claims, wherein the polypeptide comprises at least two, at least three, or at least four VHH domains that bind FcRn and at least one VHH domain that binds albumin.

41. The polypeptide of claim 40, wherein the polypeptide comprises two, three, or four VHH domains that bind FcRn and one VHH domain that binds albumin.

42. The polypeptide of any one of claims 1-41, wherein the polypeptide comprises at least one binding domain that binds a protein other than albumin or FcRn.

43. The polypeptide of claim 42, wherein at least one binding domain that binds a protein other than albumin or FcRn is a VHH.

44. The polypeptide of claim 43, wherein each binding domain that binds a protein other than albumin or FcRn is a VHH.

45. The polypeptide of claim 42 or claim 43, wherein at least one binding domain that binds a protein other than albumin or FcRn comprises a heavy chain variable region and a light chain variable region.

46. The polypeptide of claim 45, wherein each binding domain that binds a protein other than albumin or FcRn comprises a heavy chain variable region and a light chain variable region.

47. The polypeptide of any one of claims 42-46, wherein at least one binding domain that binds a protein other than albumin or FcRn is a binding domain of a therapeutic antibody.

48. The polypeptide of claim 47, wherein each binding domain that binds a protein other than albumin is a binding domain of a therapeutic antibody.

49. The polypeptide of claim 47 or claim 48, wherein the therapeutic antibody is useful for treating a disease or disorder selected from an autoimmune disease or disorder, an inflammatory disease or disorder, an infection, and cancer.

50. The polypeptide of any one of claims 1-41, wherein the polypeptide comprises an amino acid sequence of a therapeutic protein.

51. The polypeptide of claim 50, wherein the therapeutic protein is useful for treating a disease or disorder selected from an autoimmune disease or disorder, an inflammatory disease or disorder, an infection, and cancer.

52. The polypeptide of any one of the preceding claims, wherein the half-life of the polypeptide is greater than the half-life of the same polypeptide lacking a VHH domain that binds albumin.

53. A pharmaceutical composition comprising the polypeptide of any one of claims 1-52 and a pharmaceutically acceptable carrier.

54. An isolated nucleic acid that encodes the polypeptide of any one of claims 1-52.

55. A vector comprising the nucleic acid of claim 54.

56. A host cell comprising the nucleic acid of claim 54 or the vector of claim 55.

57. A host cell that expresses the polypeptide of any one of claims 1-52.

58. A method of producing the polypeptide of any one of claims 1-52, comprising incubating the host cell of claim 56 or claim 57 under conditions suitable for expression of the polypeptide.

59. The method of claim 58, further comprising isolating the antibody or polypeptide.

60. A method comprising administering to a subject the polypeptide of any one of claims 1-52, or the pharmaceutical composition of claim 53.

61. A method of treating a disease or disorder comprising administering to a subject with the disease or disorder a pharmaceutically effective amount of the polypeptide of any one of claims 1-52, or the pharmaceutical composition of claim 53.

62. The method of claim 61, wherein the disease or disorder is selected from an autoimmune disease or disorder, an inflammatory disease or disorder, an infection, and cancer.

63. The method of claim 61 or claim 62, wherein the disease or disorder is an autoantibody-mediated disease or disorder.

64. The method of any one of claims 61-63, wherein the disease or disorder is pemphigus vulgaris, lupus nephritis, myasthenia gravis, Guillain-Barré syndrome, antibody-mediated rejection, antiphospholipid antibody syndrome, chronic inflammatory demyelinating polyneuropathy, immune complex-mediated vasculitis, glomerulitis, a channelopathy, neuromyelitis optica, autoimmune encephalitis, autoimmune Grave's disease, idiopathic thrombocytopenia purpura, autoimmune haemolytic anaemia, immune neutropenia, dilated cardiomyopathy, or serum sickness.

65. The method of any one of claims 60-64, wherein the polypeptide is administered subcutaneously.
